(19) United States
(12) Patent Application Publication
(10) Pub. No.: US 2026/0274774 A1
(43) Pub. Date: Sep. 17, 2026

(54) FLUOROALCOHOLS AS CO-SOLVENTS FOR CHEMICAL SYNTHESIS AND METHODS FOR PRODUCING THE SAME

(71) Applicant: University of Mississippi, University, MS (US)

(72) Inventors: David Alan Colby, University, MS (US); Baharul Islam, University, MS (US)

(21) Appl. No.: 19/168,349

(22) PCT Filed: Mar. 21, 2024
(86) PCT No.: PCT/US2024/020876

§ 371 Date: Sep. 24, 2025

Related U.S. Application Data
(60) Provisional application No. 63/491,999, filed on Mar. 24, 2023.

(51) Int. Cl. C07C 29/143; C07C 33/46; C07C 41/26; C07C 43/23; C07D 213/26

(52) U.S. Cl. CPC C07C 29/143; C07C 33/46; C07C 41/26; C07C 43/23; C07D 213/26; C07B 2200/05; C07B 2200/07

(57) ABSTRACT

In one aspect, the disclosure relates to a method for the synthesis of penatfluoroisopropanols having a range of solubilities, acidity, basicity, and other physical properties. The method also allows the production of pentafluoroisopropanols having a chiral center. In some aspects, racemic mixtures of pentafluoroisopropanol derivatives can be separated by chiral chromatography or another method in order to isolate pure enantiomers. In one aspect, the disclosed method is compatible with a variety of pentafluoro-gem-diols and can be conducted under mild conditions with high yields. Also disclosed herein are pentafluoroisopropanols generated by the disclosed method and methods of using the pentafluoroisopropanols as solvents and co-solvents for organic synthesis, metal catalyzed reactions, asymmetric processes, carbohydrate chemistry, and medicinal chemistry.

### CROSS REFERENCE TO RELATED APPLICATIONS

This application claims the benefit of U.S. Provisional Application No. 63/491,999 filed on Mar. 24, 2023, which is incorporated herein by reference in its entirety.

### STATEMENT REGARDING FEDERALLY SPONSORED RESEARCH OR DEVELOPMENT

This invention was made with government support under grant number P20GM130460, awarded by the National Institute of General Medical Sciences of the National Institutes of Health. The government has certain rights in the invention.

### BACKGROUND

Solvent effects are critical during the discovery process in order to produce an optimal yield of product as well as enable the discovery of new chemical transformations. Moreover, efficient synthetic strategies and solvents with low environmental impact are necessities in the pharmaceutical industry. Most chemical researchers do not design new classes of solvents in assist with reaction discovery and optimization, but instead rely on small group of commonly used solvents that are well characterized. Indeed, the production of any solvent requires a route must reliably make milliliter- or liter-quantities; however, the large-scale production of any organic molecule can be a considerable challenge, especially with structures with high degrees of complexity.

The fluorination of organic compounds is a powerful strategy to tune its physical properties through the unique electronegativity of the fluorine atom. The two currently known fluorinated solvents, trifluoroethanol and hexafluoroisopropanol, have found extensive use in many chemical reactions due to high levels of hydrogen-bond donation, low nucleophilicity, and high ionizing power due to high electronegativity of the fluorine atom. Moreover, trifluoroethanol has now been integrated into the multi-gram and multi-kilogram production of active pharmaceutical ingredients, which is a necessity for the healthcare field to treat diseases and conduct clinical trials. At the present, researchers will screen these two fluorinated alcohols to improve a reaction; however, in some cases, they both fail, and it is not clear why. Trifluoroethanol and hexafluoroisopropanol are achiral compounds and are limited in physical properties, thus also limiting their usefulness as solvents or co-solvents.

Although synthetic methods have been widely developed for the creation of fluorinated and trifluoromethylated structures, methods for many other classes of highly fluorinated structures are under-developed. This limitation has restricted the potential of using fluorinated alcohols to discover new reactions, because trifluoroethanol, hexafluoroisopropanol, as well as a few new fluorinated solvents, all display only trifluoromethyl groups.

Recent reports have demonstrated that using fluorinated alcohols as solvents promote state-of-the-art bond-activation and photoredox catalysis reactions whereas these processes fail when using the non-fluorinated counterparts. Fluorinated alcohols clearly enable many other chemical reactions such as C—C bond forming reactions, cyclizations, and solvolysis, but the mechanisms for this behavior are not well understood. The discovery of new classes of fluorinated alcohols to elucidate the mechanisms of the behavior and enable the design of new chemical reactions is needed to advance the field of organic synthesis and the production of pharmaceuticals.

The incorporation of fluorine atoms on organic molecules is a common objective during drug development. Synthetic methods for the fluorination and trifluoromethylation of compounds are significantly more developed than strategies that create a difluoromethyl group. A mild process for the generation of α,α-difluoroenolates from the fragmentation of pentafluoro-gem-diols has previously been developed. An expansion of the reactivity of pentafluoro-gem-diols could potentially access to valuable pentafluoroisopropanols and open new avenues for the synthesis of additional, multiply-fluorinated targets.

Despite advances in synthetic research, there is still a scarcity of fluoroalcohols with wide-ranging and customizable physical properties that are useful as solvents or co-solvents for pharmaceuticals, fine chemicals, and other fluorochemicals. Ideally, new fluoroalcohols could be synthesized under mild conditions and with few or no undesirable side products. In some aspects, the new fluoroalcohols could have chiral centers and be present as either a racemic mixture or could be separated into enantiomers, enabling further applications as solvents or co-solvents for chiral starting materials and/or products. These needs and other needs are satisfied by the present disclosure.

### SUMMARY

In accordance with the purpose(s) of the present disclosure, as embodied and broadly described herein, the disclosure, in one aspect, relates to a method for the synthesis of penatfluoroisopropanols having a range of solubilities, acidity, basicity, and other physical properties. The method also allows the production of pentafluoroisopropanols having a chiral center. In some aspects, racemic mixtures of pentafluoroisopropanol derivatives can be separated by chiral chromatography or another method in order to isolate pure enantiomers. In one aspect, the disclosed method is compatible with a variety of pentafluoro-gem-diols and can be conducted under mild conditions with high yields. Also disclosed herein are pentafluoroisopropanols generated by the disclosed method and methods of using the pentafluoroisopropanols as solvents and co-solvents for organic synthesis, metal catalyzed reactions, asymmetric processes, carbohydrate chemistry, and medicinal chemistry.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows fluorine NMR of the crude mixture of a reaction converting a pentafluoro-gem-diol to a pentafluoroisopropanol using pyridine with CeCl₃·7H₂O and NaBH₄.

FIG. 2 shows fluorine NMR of the crude mixture of a reaction converting a pentafluoro-gem-diol to a pentafluoroisopropanol using tert-butanol with CeCl₃·7H₂O and NaBH₄.

FIG. 3 shows proton NMR of air-dried sample containing an exemplary pentafluoroisopropanol after purification.

FIG. 4 shows proton NMR of an exemplary sample after rotary evaporation displaying no signals, which is an indication of compound's volatility.

### DETAILED DESCRIPTION

Trifluoroethanol and hexafluoroisopropanol (i.e., TFE and HFIP, respectively) enable many chemical reactions such as carbon-carbon bond forming reactions, cyclizations, and solvolysis. Also, trifluoroethanol, has been integrated into the multi-gram and kilogram scale production of active pharmaceutical ingredients, which is a necessity for the healthcare field to treat diseases and conduct clinical trials. Even though these two fluorinated alcohols have been used to discover new reactions and optimize existing ones, in some cases, they both fail, and it is not clear why. Prior studies on the mechanisms of trifluoroethanol and hexafluoroisopropanol have investigated how changing the structure of the starting material affects the formation of products, but the development of new fluorinated alcohols with structures that tune the desirable properties of hydrogen bonding, nucleophilicity, and ionization remains largely unaddressed. Indeed, a larger array of fluoroalcohols will help solve these mechanistic puzzles.

The existing analogues of trifluoroethanol and hexafluoroisopropanol are primarily derived from exchanging a hydrogen atom adjacent to the hydroxyl group with an alkyl or aromatic ring. These products are commonly derived from trifluoromethylation of aldehydes or nucleophilic addition to hexafluoroacetone. However, the fundamental classification of the alcohol has changed, because the adducts of trifluoroethanol are secondary alcohols whereas those of hexafluoroisopropanol are tertiary alcohols. This modification to the structure of the alcohol is not subtle, because chemical reactivity and properties are well known to change dynamically across primary, secondary, and tertiary alcohols. Indeed, this issue is essential because recent advances in catalysis have proposed the participation of fluorinated alcohols in palladium, calcium, magnesium, and molybdenum complexes. For example, hexafluoroisopropanol has been observed by NMR in the active complex with palladium during C—H activation. It can be envisioned from the tight complex formed that replacing hexafluoroisopropanol with a larger tertiary alcohol, such as hexafluoro-2-methylisopropanol or hexafluoro-2-phenylisopropanol, will not fit into the complex, and in turn, reduce the efficiency of the reaction. Thus, the design of new fluoroalcohols must factor in the potential to exploit, not hinder, this new development.

Disclosed herein is a method for making a substituted pentafluoroisopropanol, the method including at least the steps of contacting a pentafluoro-gem-diol of Formula I with a reducing agent:

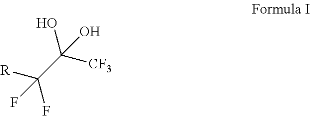

wherein R is a substituted or unsubstituted aryl or heteroaryl group having Formula IIa or IIb, a ketone having Formula III, a C₃-C₆ substituted or unsubstituted cycloalkyl or heterocycloalkyl group, a C₁-C₅ linear or branched alkane, a substituted or unsubstituted ether, a substituted or unsubstituted amide, a substituted or unsubstituted ester, an acetyl group, or a substituted or unsubstituted sulfoxide;

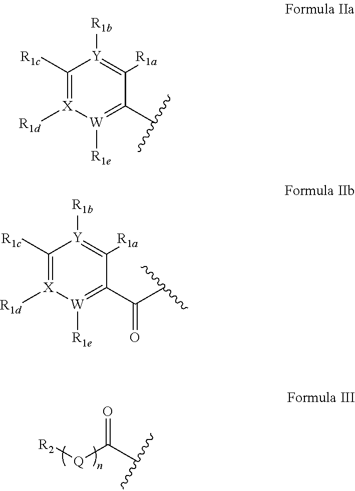

wherein, in Formula IIa or IIb, W, X, and Y are independently C or N;
wherein R₁ₐ and R_{1c} are independently selected from hydrogen, nitro, halogen, cyano, trifluoromethyl, hydroxyl, linear or branched C₁-C₁₀ alkyl, or any combination thereof;
wherein, when Y is C, R_{1b} is selected from hydrogen, nitro, halogen, cyano, trifluoromethyl, hydroxyl, linear or branched C₁-C₁₀ alkyl, or any combination thereof and when Y is N, R_{1b} is absent;
wherein, when X is C, R_{1d} is selected from hydrogen, nitro, halogen, cyano, trifluoromethyl, hydroxyl, linear or branched C₁-C₁₀ alkyl, or any combination thereof and when X is N, R_{1d} is absent; and
wherein, when W is C, R₁ₑ is selected from hydrogen, nitro, halogen, cyano, trifluoromethyl, hydroxyl, linear or branched C₁-C₁₀ alkyl, or any combination thereof and when W is N, R₁ₑ is absent;
wherein, in Formula III, n is from 0 to 10;
wherein when n is 1, Q is —CH₂—, and when n is from 2 to 10, Q is —CH₂— or —CH—; and
wherein R₂ is selected from a substituted or unsubstituted C₆-C₁₀ aryl or heteroaryl group, an adamantyl group, or any combination thereof.

In some aspects, in Formula IIa or IIb, X and Y are C and W is N or C. In one aspect, W is C, R₁ₐ, R_{1b}, and R_{1d} are H, and R_{1c} and R₁ₑ are independently selected from CF₃, NO₂, H, and methyl. In an alternative aspect, W is N, R₁ₐ and R_{1b} are H, and R_{1c} and R_{1d} are independently selected from CF₃, NO₂, H, and methyl. In an aspect, R is C₁ to C₅ linear alkyl. In some aspects, n is from 0 to 2.

In one aspect, the pentafluoro-gem-diol of Formula I can be selected from:

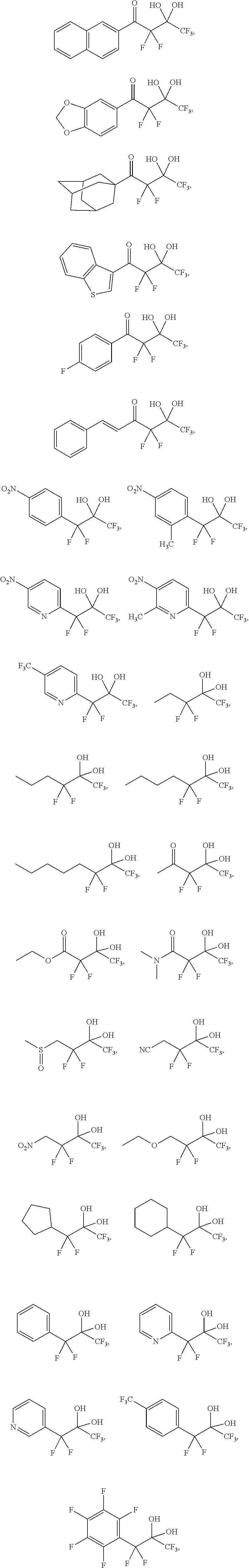

or any combination thereof.

In one aspect, the reducing agent can be NaBH₄, LiBH₄, LiAlH₄, diisobutylaluminum hydride (DIBALH), or any combination thereof. In an aspect, in order to produce a deuterated hexafluoroisopropanol, the reducing agent can be deuterated, such as, for example, NaBD₄, LiBD₄, LiAlD₄, diisobutylaluminum deuteride, or any combination thereof.

In another aspect, the method can further include contacting the pentafluoro-gem-diol of Formula I with CsCl₃ or a hydrate thereof. In still another aspect, the method can further include contacting the pentafluoro-gem-diol of Formula I with a basic additive such as, for example, triethylamine (Et₃N), 1,8-diazabicyclo[5.4.0]undec-7-ene (DBU), 1,5-diazabicyclo[4.3.0]non-5-ene (DBN), collidine, lutidine, N,N-diisopropylethylamine (iPr₂NEt), or any combination thereof.

In one aspect, the method can be performed at a temperature of form about 10° C. to about 60° C., or at about 10, 15, 20, 25, 30, 35, 40, 45, 50, 55, or about 60° C., or a combination of any of the foregoing values, or a range encompassing any of the foregoing values. In one aspect, the method can be performed under an inert atmosphere such as, for example, argon.

In another aspect, the substituted pentafluoroisopropanol has Formula IV:

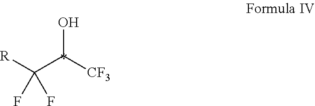

wherein the substituted pentafluoroisopropanol has R stereochemistry, S stereochemistry, or a combination of R and S stereochemistry at a carbon atom indicated by *;
wherein R comprises a substituted or unsubstituted aryl or heteroaryl group having Formula IIa or Formula IIb or a ketone having Formula III, a C₃-C₆ substituted or unsubstituted cycloalkyl or heterocycloalkyl group, a C₁-C₅ linear or branched alkane, a substituted or unsubstituted ether, a substituted or unsubstituted amide, a substituted or unsubstituted ester, an acetyl group, or a substituted or unsubstituted sulfoxide;

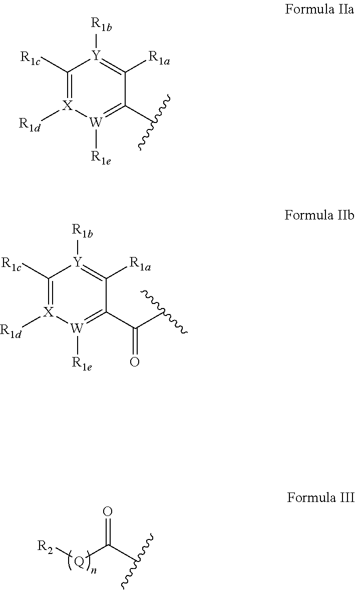

wherein, in Formula IIa and Formula IIb, W, X, and Y are independently C or N;
wherein R₁ₐ and R_{1c} are independently selected from hydrogen, nitro, halogen, cyano, trifluoromethyl, hydroxyl, linear or branched C₁-C₁₀ alkyl, or any combination thereof;
wherein, when Y is C, R_{1b} is selected from hydrogen, nitro, halogen, cyano, trifluoromethyl, hydroxyl, linear or branched C₁-C₁₀ alkyl, or any combination thereof and when Y is N, R_{1b} is absent;
wherein, when X is C, R_{1d} is selected from hydrogen, nitro, halogen, cyano, trifluoromethyl, hydroxyl, linear or branched C₁-C₁₀ alkyl, or any combination thereof and when X is N, R_{1d} is absent; and
wherein, when W is C, R₁ₑ is selected from hydrogen, nitro, halogen, cyano, trifluoromethyl, hydroxyl, linear or branched C₁-C₁₀ alkyl, or any combination thereof and when W is N, R₁ₑ is absent;
wherein, in Formula III, n is from 0 to 10;
wherein when n is 1, Q is —CH₂—, and when n is from 2 to 10, Q is —CH₂— or —CH—; and
wherein R₂ is selected from a substituted or unsubstituted C₆-C₁₀ aryl or heteroaryl group, an adamantyl group, or any combination thereof.

In one aspect, also disclosed are substituted pentafluoroisopropanols made by the disclosed method. In some aspects, in Formula IIa or IIb, X and Y are C and W is N or C. In one aspect, W is C, R₁ₐ, R_{1b}, and R_{1d} are H, and R_{1c} and R₁ₑ are independently selected from CF₃, NO₂, H, and methyl. In an alternative aspect, W is N, R₁ₐ and R_{1b} are H, and R_{1c} and R_{1d} are independently selected from CF₃, NO₂, H, and methyl. In an aspect, R is C₁ to C₅ linear alkyl. In some aspects, n is from 0 to 2.

In another aspect, the substituted pentafluoroisopropanol can have the structure:

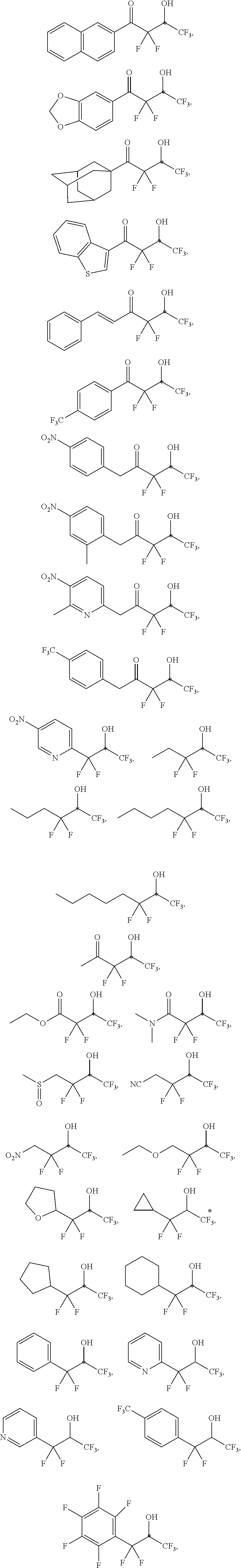

or any combination thereof.

In one aspect, the disclosed pentafluoroisopropanols are useful as solvents or co-solvents, or components of solvents or co-solvents, or as catalysts for organic synthesis. Further in this aspect, the solvents and co-solvents can be used to produce agrochemicals, polymers, petrochemicals, pharmaceuticals, and other fine chemicals, and can be used in the fabrication of electronic components including motherboards, microprocessors, and the like.

In one aspect, the substituted pentafluoroisopropanol includes at least one deuterium atom in place of at least one hydrogen atom. In another aspect, the substituted pentafluoroisopropanol can be fully deuterated.

In one aspect, the substituted pentafluoroisopropanol has an enantiomeric excess of from about 5% to about 95%, or about 5, 10, 15, 20, 25, 30, 35, 40, 45, 50, 55, 60, 65, 70, 75, 80, 85, 90, or about 95% of an R enantiomer at the carbon atom indicated by *, or has an enantiomeric excess of from about 5% to about 95%, or about 5, 10, 15, 20, 25, 30, 35, 40, 45, 50, 55, 60, 65, 70, 75, 80, 85, 90, or about 95% of an S enantiomer at the carbon atom indicated by *. In some aspects, the substituted pentafluoroisopropanol is present as a racemic mixture.

In still another aspect, disclosed herein is a method for making an enantiomerically pure substituted pentafluoroisopropanol, the method including at least the step of separating a mixture of enantiomers of the disclosed substituted pentafluoroisopropanol using chiral chromatography.

In one aspect, disclosed herein is a solvent or co-solvent for organic synthesis including a disclosed substituted isopropanol. In some aspects, the solvent or co-solvent has a lower boiling point than an otherwise identical solvent bearing one or more H atoms in place of F atoms.

An exemplary reaction to produce a substituted pentafluoroisopropanol is shown in Scheme 1:

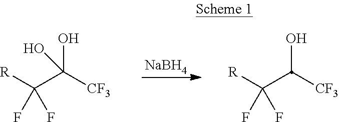

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a carbonate base,” “a source of formaldehyde,” or “a difluoroenolate anion,” include, but are not limited to, mixtures or combinations of two or more such carbonate bases, sources of formaldehyde, or difluoroenolate anions, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y.’ The range can also be expressed as an upper limit, e.g. ‘about x, y, z, or less’ and should be interpreted to include the specific ranges of ‘about x,’ ‘about y’, and ‘about z’ as well as the ranges of ‘less than x’, less than y’, and ‘less than z’. Likewise, the phrase ‘about x, y, z, or greater’ should be interpreted to include the specific ranges of ‘about x,’ ‘about y,’ and ‘about z’ as well as the ranges of ‘greater than x,’ greater than y,’ and ‘greater than z.’ In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched, and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C₁ alkyl, C₁-C₂ alkyl, C₁-C₃ alkyl, C₁-C₄ alkyl, C₁-C₅ alkyl, C₁-C₆ alkyl, C₁-C₇ alkyl, C₁-C₈ alkyl, C₁-C₉ alkyl, C₁-C₁₀ alkyl, and the like up to and including a C₁-C₂₄ alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂-(methylene), —CH₂CH₂—, —CH₂C (CH₃)₂CH₂—, and —CH₂CH₂CH₂— are non-limiting examples of alkanediyl groups.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C (A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²⁰)ₐ-, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘}₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘)}₂; —(CH₂)₀₋₄N(R^{∘})C(O)R^{∘}; —N(R^{∘})C(S)R^{∘}; —(CH₂)₀₋₄N(R^{∘})C(O)NR^{∘})₂; —N(R^{∘})C(S)NR^{∘})₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘})₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O) SR^{∘}; —(CH₂)₀₋₄C(O)OSiR^{∘})₃; —(CH₂)₀₋₄OC(O)R^{∘}; —OC(O) (CH₂)₀₋₄SR—, SC(S)SR^{∘}; —(CH₂)₀₋₄SC(O)R^{∘}; —(CH₂)₀₋₄C(O)NR^{∘})₂; —C(S)NR^{∘})₂; —C(S)SR^{∘}; —(CH₂)₀₋₄OC(O)NR^{∘})₂; —C(O)N(OR^{∘})R^{∘}; —C(O)C(O)R^{∘}; —C(O) CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘})₂; —(CH₂)₀₋₄S(O)R^{∘}; —N(R^{∘}S(O)₂NR^{∘})₂; —N(R^{∘}S(O)₂R^{∘}; —N(OR^{∘})R^{∘}; —C(NH)NR^{∘})₂; —P(O)₂R^{∘}; —P(O)R^{∘)}₂; —OP(O)R^{∘)}₂; —OP(O) (OR^{∘})₂; SiR^{∘}₃; —(C₁₋₄ straight or branched alkylene)O—N(R)₂; or —(C₁₋₄ straight or branched alkylene)C(O)O—N(R)₂, wherein each R^{∘} may be substituted as defined below and is independently hydrogen, C₁₋₆ aliphatic, —CH₂Ph, —O(CH₂)₀₋₁Ph, —CH₂—(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•})₂, —NO₂, —SiR^{•}3, —OSiR^{•})₃, —C(O) SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR^{*}₂, ═NNHC(O)R^{*}, ═NNHC(O)OR^{*}, ═NNHS(O)₂R^{*}, ═NR^{*}, ═NOR^{*}, —O(C(R^{*}₂))₂₋₃O—, or —S(C(R^{*}₂))₂₋₃S—, wherein each independent occurrence of R^{*} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include:—O(CR^{*}₂)₂₋₃O—, wherein each independent occurrence of R^{*} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{*} include halogen, —R^{•}), -(haloR^{•}, —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}2, —C(O)R^{†}, —C(O)OR^{†}, —C(O)C(O)R^{†}, —C(O) CH₂C(O)R^{†}, —S(O)₂R^{†}, —S(O)₂NR^{†}2, —C(S)NR^{†}2, —C(NH)NR^{†}2, or —N(R^{†}) S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{∘})₂, or —NO₂, wherein each R^{∘} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolyzable group” and “hydrolyzable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

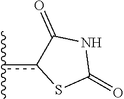

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms, and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and I or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and(S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or(S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine, and chlorine, such as ²H, ³H, ¹³C, ¹⁴C, ¹⁵N, ¹⁸O, ¹⁷O, ³⁵S, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

It is also appreciated that certain compounds described herein can be present as an equilibrium of tautomers. For example, ketones with an α-hydrogen can exist in an equilibrium of the keto form and the enol form.

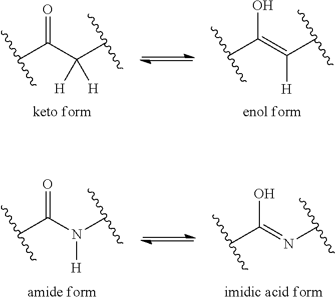 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

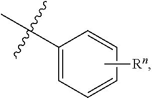

which is understood to be equivalent to a formula:

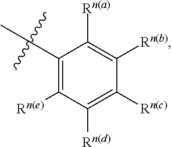

wherein n is typically an integer. That is, Rⁿ is understood to represent five independent substituents, R^{n(a)}, R^{n(b)}, R^{n(c)}, R^{n(d)}, and R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

A “solvent” as used herein refers to a substance that dissolves at least one or more solute, resulting in a solution. In one aspect, one or more solutes dissolved in a solvent can undergo a chemical reaction to produce a new substance. In a still further aspect, the pentafluoroisopropanols disclosed herein are useful as solvents in a variety of chemical processes as disclosed herein and may be particularly useful for reactions involving specific bond activation, photoredox catalysis, cyclization, solvolysis, and the like.

Meanwhile, a “co-solvent” as used herein refers to a substance added to a solvent in a small amount to increase the solubility of a poorly-soluble compound. In some aspects, co-solvents can be particularly useful to overcome hydrophobicity of poorly-soluble compounds, allowing their dissolution in aqueous solutions. In one aspect, the disclosed pentafluoroisopropanols are useful as co-solvents for producing and formulating pharmaceuticals, agrochemicals, petrochemicals, and the like.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

In various aspects, the disclosed compounds can possess at least one center of asymmetry, they can be present in the form of their racemates, in the form of the pure enantiomers and/or diastereomers or in the form of mixtures of these enantiomers and/or diastereomers. The stereoisomers can be present in the mixtures in any arbitrary proportions. In some aspects, provided this is possible, the disclosed compounds can be present in the form of the tautomers.

Thus, methods which are known per se can be used, for example, to separate the disclosed compounds which possess one or more chiral centers and occur as racemates into their optical isomers, i.e., enantiomers or diastereomers. The separation can be effected by means of column separation on chiral phases or by means of recrystallization from an optically active solvent or using an optically active acid or base or by means of derivatizing with an optically active reagent, such as an optically active alcohol, and subsequently cleaving off the residue.

Unless otherwise specified, pressures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

The present disclosure can be described in accordance with the following numbered aspects, which should not be confused with the claims.

Aspect 1. A method for making a substituted pentafluoroisopropanol, the method comprising contacting a pentafluoro-gem-diol of Formula I with a reducing agent:

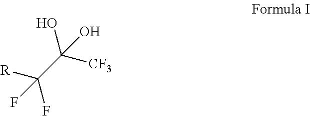

wherein R comprises a substituted or unsubstituted aryl or heteroaryl group having Formula IIa or IIb, a ketone having Formula III, a C₃-C₆ substituted or unsubstituted cycloalkyl or heterocycloalkyl group, a C₁-C₅ linear or branched alkane, a substituted or unsubstituted ether, a substituted or unsubstituted amide, a substituted or unsubstituted ester, an acetyl group, or a substituted or unsubstituted sulfoxide;

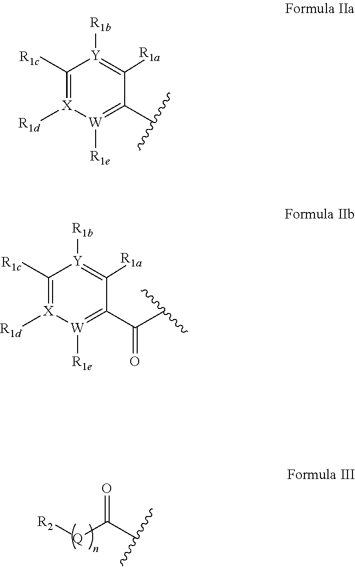

wherein, in Formula IIa or IIb, W, X, and Y are independently C or N;
wherein R₁ₐ and R_{1c} are independently selected from hydrogen, nitro, halogen, cyano, trifluoromethyl, hydroxyl, linear or branched C₁-C₁₀ alkyl, or any combination thereof;
wherein, when Y is C, R_{1b} is selected from hydrogen, nitro, halogen, cyano, trifluoromethyl, hydroxyl, linear or branched C₁-C₁₀ alkyl, or any combination thereof and when Y is N, R_{1b} is absent;
wherein, when X is C, R_{1d} is selected from hydrogen, nitro, halogen, cyano, trifluoromethyl, hydroxyl, linear or branched C₁-C₁₀ alkyl, or any combination thereof and when X is N, R_{1d} is absent; and
wherein, when W is C, R₁ₑ is selected from hydrogen, nitro, halogen, cyano, trifluoromethyl, hydroxyl, linear or branched C₁-C₁₀ alkyl, or any combination thereof and when W is N, R₁ₑ is absent;
wherein, in Formula III, n is from 0 to 10;
wherein when n is 1, Q is —CH₂—, and when n is from 2 to 10, Q is —CH₂— or —CH—; and
wherein R₂ is selected from a substituted or unsubstituted C₆-C₁₀ aryl or heteroaryl group, an adamantyl group, or any combination thereof.

Aspect 2. The method of aspect 1, wherein in Formula IIa or IIb, X and Y are C and Wis N or C.

Aspect 3. The method of aspect 2, wherein W is C, wherein R₁ₐ, R_{1b}, and R_{1d} are H, and wherein R_{1c} and R₁ₑ are independently selected from CF₃, NO₂, H, and methyl.

Aspect 4. The method of aspect 2, wherein W is N, wherein R₁ₐ and R_{1b} are H, and wherein R_{1c} and R_{1d} are independently selected from CF₃, NO₂, H, and methyl.

Aspect 5. The method of aspect 1, wherein R is C₁ to C₅ linear alkyl.

Aspect 6. The method of aspect 1, wherein n is from 0 to 2.

Aspect 7. The method of any one of aspects 1-6, wherein the pentafluoro-gem-diol of Formula I is selected from:

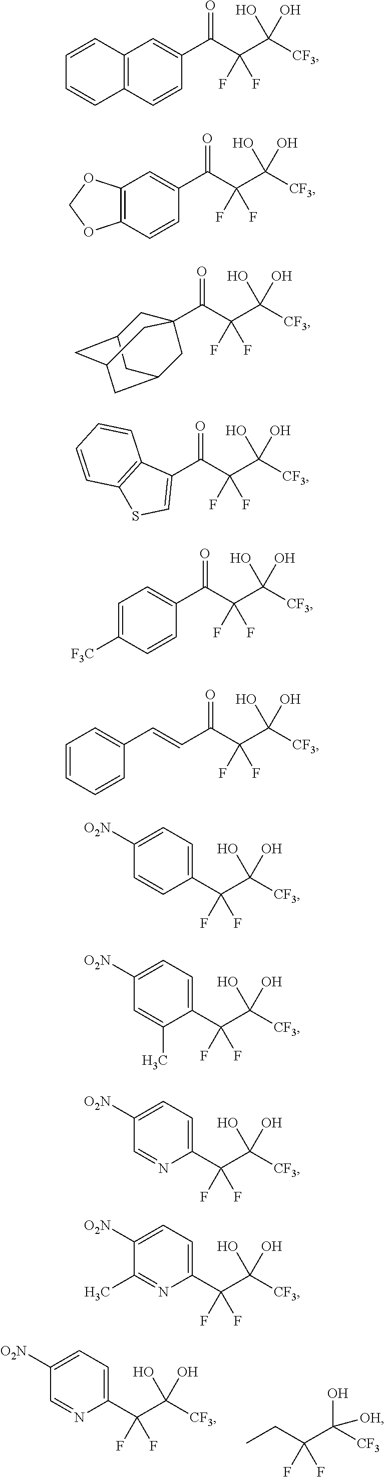

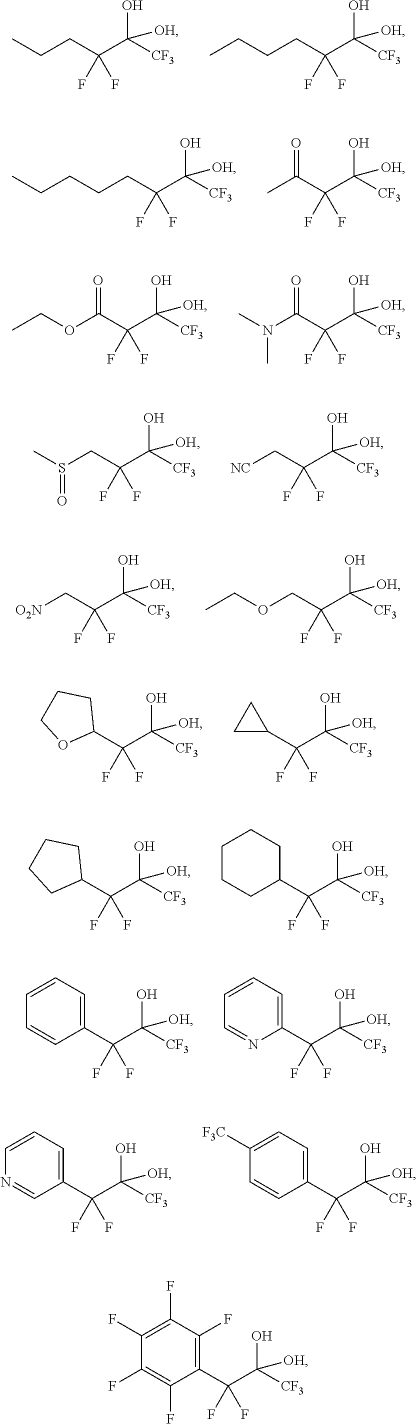

or any combination thereof.

Aspect 8. The method of any one of aspects 1-7, wherein the reducing agent comprises NaBH₄, LiBH₄, LiAlH₄, diisobutylaluminum hydride (DIBALH), or any combination thereof.

Aspect 9. The method of any one of aspects 1-8, further comprising contacting the pentafluoro-gem-diol of Formula I with CsCl₃ or a hydrate thereof.

Aspect 10. The method of any one of aspects 1-9, further comprising contacting the pentafluoro-gem-diol of Formula I with a basic additive.

Aspect 11. The method of aspect 10, wherein the basic additive comprises triethylamine (Et₃N), 1,8-diazabicyclo[5.4.0]undec-7-ene (DBU), 1,5-diazabicyclo[4.3.0]non-5-ene (DBN), collidine, lutidine, N, N-diisopropylethylamine (iPr₂NEt), or any combination thereof.

Aspect 12. The method of any one of aspects 1-11, wherein the method is performed at a temperature of from about 10° C. to about 60° C.

Aspect 13. The method of any one of aspects 1-12, wherein the method is performed under argon.

Aspect 14. The method of any one of aspects 1-13, wherein the substituted pentafluoroisopropanol has Formula IV:

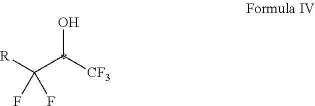

wherein the substituted pentafluoroisopropanol has R stereochemistry, S stereochemistry, or a combination of R and S stereochemistry at a carbon atom indicated by *;
wherein R comprises a substituted or unsubstituted aryl or heteroaryl group having Formula IIa or Formula IIb or a ketone having Formula III, a C₃-C₆ substituted or unsubstituted cycloalkyl or heterocycloalkyl group, a C₁-C₅ linear or branched alkane, a substituted or unsubstituted ether, a substituted or unsubstituted amide, a substituted or unsubstituted ester, an acetyl group, or a substituted or unsubstituted sulfoxide:

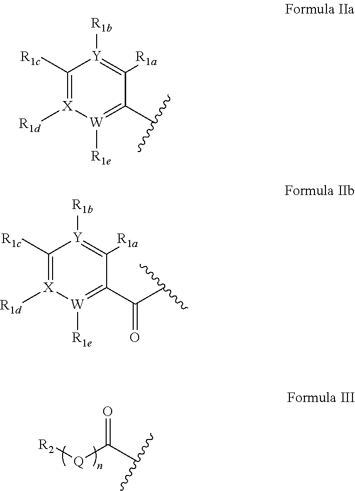

wherein, in Formula IIa and Formula IIb, W, X, and Y are independently C or N;
wherein R₁ₐ and R_{1c} are independently selected from hydrogen, nitro, halogen, cyano, trifluoromethyl, hydroxyl, linear or branched C₁-C₁₀ alkyl, or any combination thereof;
wherein, when Y is C, R_{1b} is selected from hydrogen, nitro, halogen, cyano, trifluoromethyl, hydroxyl, linear or branched C₁-C₁₀ alkyl, or any combination thereof and when Y is N, R_{1b} is absent;
wherein, when X is C, R_{1d} is selected from hydrogen, nitro, halogen, cyano, trifluoromethyl, hydroxyl, linear or branched C₁-C₁₀ alkyl, or any combination thereof and when X is N, R_{1d} is absent; and
wherein, when W is C, R₁ₑ is selected from hydrogen, nitro, halogen, cyano, trifluoromethyl, hydroxyl, linear or branched C₁-C₁₀ alkyl, or any combination thereof and when W is N, R₁ₑ is absent;
wherein, in Formula III, n is from 0 to 10;
wherein when n is 1, Q is —CH₂—, and when n is from 2 to 10, Q is —CH₂— or —CH—; and
wherein R₂ is selected from a substituted or unsubstituted C₆-C₁₀ aryl or heteroaryl group, an adamantyl group, or any combination thereof.

Aspect 15. A substituted pentafluoroisopropanol produced by the method of any one of aspects 1-14.

Aspect 16. A substituted pentafluoroisopropanol having Formula IV:

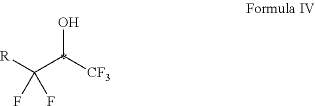

wherein the substituted pentafluoroisopropanol has R stereochemistry, S stereochemistry, or a combination of R and S stereochemistry at a carbon atom indicated by *;
wherein R comprises a substituted or unsubstituted aryl or heteroaryl group having Formula IIa or Formula IIb or a ketone having Formula III, a C₃-C₆ substituted or unsubstituted cycloalkyl or heterocycloalkyl group, a C₁-C₅ linear or branched alkane, a substituted or unsubstituted ether, a substituted or unsubstituted amide, a substituted or unsubstituted ester, an acetyl group, or a substituted or unsubstituted sulfoxide;

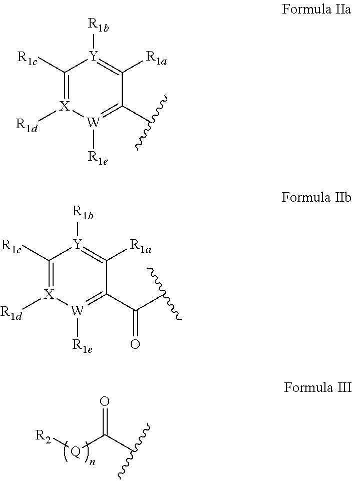

wherein, in Formula IIa and Formula IIb, W, X, and Y are independently C or N;
wherein R₁ₐ and R_{1c} are independently selected from hydrogen, nitro, halogen, cyano, trifluoromethyl, hydroxyl, linear or branched C₁-C₁₀ alkyl, or any combination thereof;
wherein, when Y is C, R_{1b} is selected from hydrogen, nitro, halogen, cyano, trifluoromethyl, hydroxyl, linear or branched C₁-C₁₀ alkyl, or any combination thereof and when Y is N, R_{1b} is absent;
wherein, when X is C, R_{1d} is selected from hydrogen, nitro, halogen, cyano, trifluoromethyl, hydroxyl, linear or branched C₁-C₁₀ alkyl, or any combination thereof and when X is N, R_{1d} is absent; and wherein, when W is C, R₁ₑ is selected from hydrogen, nitro, halogen, cyano, trifluoromethyl, hydroxyl, linear or branched C₁-C₁₀ alkyl, or any combination thereof and when W is N, R₁ₑ is absent;
wherein, in Formula III, n is from 0 to 10;
wherein when n is 1, Q is —CH₂—, and when n is from 2 to 10, Q is —CH₂— or —CH—; and
wherein R₂ is selected from a substituted or unsubstituted C₆-C₁₀ aryl or heteroaryl group, an adamantyl group, or any combination thereof.

Aspect 17. The substituted pentafluoroisopropanol of aspect 16, wherein in Formula IIa and Formula IIb, X and Y are C and W is N or C.

Aspect 18. The substituted pentafluoroisopropanol of aspect 17, wherein W is C, wherein R₁ₐ, R_{1b}, and R_{1d} are H, and wherein R_{1c} and R₁ₑ are independently selected from CF₃, NO₂, H, and methyl.

Aspect 19. The substituted pentafluoroisopropanol of aspect 17, wherein W is N, wherein R₁ₐ and Rib are H, and wherein R_{1c} and R₁ₐ are independently selected from CF₃, NO₂, H, and methyl.

Aspect 20. The substituted pentafluoroisopropanol of aspect 16, wherein R is a C₁ to C₅ linear alkyl.

Aspect 21. The substituted pentafluoroisopropanol of aspect 16, wherein n is from 0 to 2.

Aspect 22. The substituted pentafluoroisopropanol of any one of aspects 16-21, having the formula:

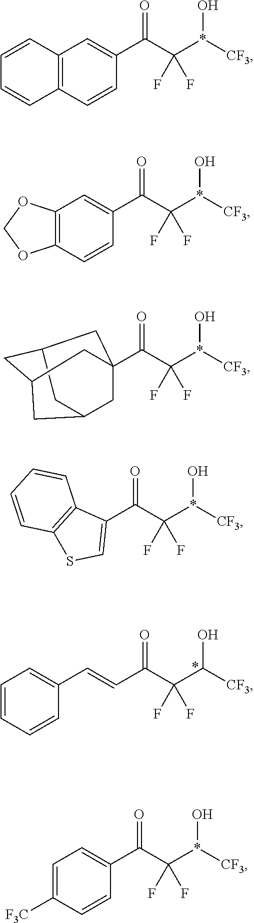 [SMILES: O=C(c1ccc2ccccc2c1)C(F)(F)*(O)C(F)(F)F ; O=C(c1ccc2c(c1)OCO2)C(F)(F)*(O)C(F)(F)F ; O=C(C12CC3CC(CC(C3)C1)C2)C(F)(F)*(O)C(F)(F)F ; O=C(c1csc2ccccc12)C(F)(F)*(O)C(F)(F)F ; O=C(/C=C/c1ccccc1)C(F)(F)C(O)C(F)(F)F ; O=C(c1ccc(C(F)(F)F)cc1)C(F)(F)*(O)C(F)(F)F]

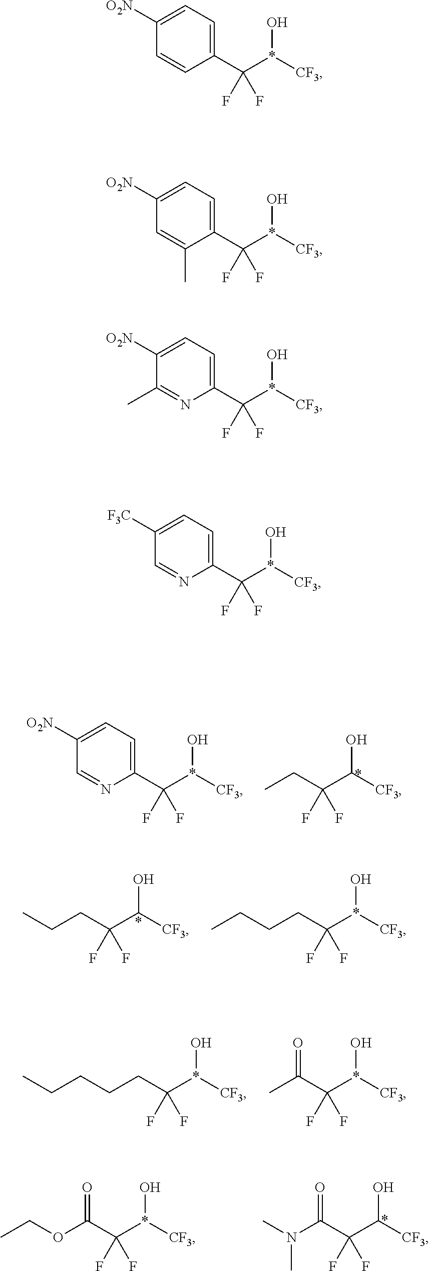

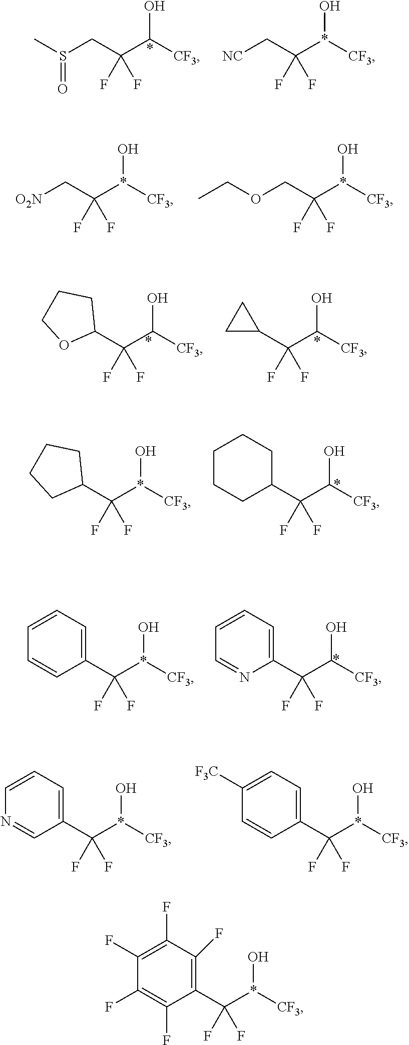

or any combination thereof.

Aspect 23. The substituted pentafluoroisopropanol of any one of aspects 16-22, wherein the substituted pentafluoroisopropanol comprises at least one deuterium atom in place of at least one hydrogen atom.

Aspect 24. The substituted pentafluoroisopropanol of aspect 23, wherein the substituted pentafluoroisopropanol is fully deuterated.

Aspect 25. The substituted pentafluoroisopropanol of any one of aspects 16-24, wherein the substituted pentafluoroisopropanol has an enantiomeric excess of from about 5% to about 95% of an R enantiomer at the carbon atom indicated by *.

Aspect 26. The substituted pentafluoroisopropanol of any one of aspects 16-24, wherein the substituted pentafluoroisopropanol has an enantiomeric excess of from about 5% to about 95% of an S enantiomer at the carbon atom indicated by *.

Aspect 27. The substituted pentafluoroisopropanol of any one of aspects 16-24, wherein the substituted pentafluoroisopropanol is present as a racemic mixture.

Aspect 28. A solvent or co-solvent for organic synthesis comprising the substituted pentafluoroisopropanol of any one of aspects 16-27.

Aspect 29. The solvent or co-solvent of aspect 28, wherein the solvent or co-solvent has a lower boiling point than an otherwise identical solvent bearing one or more H atoms in place of F atoms.

Aspect 30. A method for making an enantiomerically pure substituted pentafluoroisopropanol, the method comprising separating a mixture of enantiomers of the substituted pentafluoroisopropanol of any one of aspects 16-29 using chiral chromatography.

Aspect 31. An enantiomerically pure substituted pentafluoroisopropanol produced by the method of aspect 30.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: General Procedures

Experiments requiring anhydrous conditions were performed under argon atmosphere and organic solvents were dried over molecular sieves. All solvents and reagents were purchased from commercial sources unless otherwise noted. Gem-diols used as starting materials for compounds 1-5 and 13-18 were prepared according to previously published methods. Thin-layer chromatography was conducted using MilliporeSigma TLC silica gel 60 F254 plates. Preparative thin-layer chromatography was performed using Sorbent Technologies silica G prep TLC plates with UV254. Flash chromatography was conducted using SiliCycle Siliaflash silica gel P60 (40-63 μm) 60 Å. Melting points were taken on an OptiMelt apparatus from Stanford Research Systems and are not corrected. NMR spectra were recorded on a Bruker ARX 300 MHz, a Bruker Topspin Avance III HD 500 MHz spectrometer equipped with prodigy cryoprobe, or a Bruker Avance III HD 400 MHz spectrometer. The residual solvent peaks were used as an internal standard for ¹H and ¹³C NMR spectra, while trifluorotoluene was used as an added internal standard for ¹⁹F NMR spectra. Mass spectra were acquired by the Department of Chemistry at the University of Mississippi using SYNAPT HD Mass Spectrometer from Waters. Infrared spectra were recorded on Agilent Technologies Cary 630 FTIR.

### Example 2: Procedure to Reduce Pentafluoro-gem-Diol to Pentafluoro-Propan-2-ol

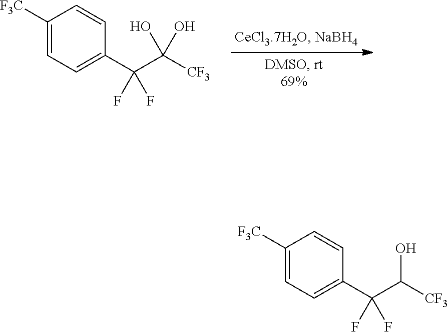 [SMILES: OC(O)(C(F)(F)F)C(F)(F)c1ccc(C(F)(F)F)cc1 ; OC(C(F)(F)F)C(F)(F)c1ccc(C(F)(F)F)cc1]

1,1,1,3,3-Pentafluoro-3-(4-(trifluoromethyl)phenyl) propan-2-ol. To a solution of CeCl₃·7H₂O(75 mg, 0.2 mmol) and gem-diol (31 mg, 0.1 mmol) in DMSO (1.0 mL) was added NaBH₄ (19 mg, 0.5 mmol) at rt. The reaction mixture was then stirred for 2 hours at rt. The yield was quantified to be 69% calculated with 19F-NMR using trifluorotoluene as a standard. 1H NMR (400 MHZ, CDCl₃) δ 7.74 (d, J=8.3 Hz, 2H), 7.69 (d, J=8.5 Hz, 2H), 4.52-4.36 (m, 1H), 2.89 (d, J=7.9 Hz, 1H); ¹⁹F NMR (376 MHZ, CDCl₃) 0-62.94 (3F), −73.40 (td, J_{FF}=10.7, J_{HF}=6.8 Hz, 3F), −100.59 (dp, J_{FF}=262.7, J_{HF}=10.0 Hz, 1F), −109.19 (dp, J_{FF}=262.6, J_{HF}=11.3 Hz, 1F); HRMS (ESI-TOF) m/z calcd for C₁₀H₅F₈O [M-H]⁻ 293.0213, found 293.0191.

### Example 3: Additional Routes to Pentafluoroisopropanols

Our strategy is partially inspired from previous work of in which hexafluoroisopropanol was fragmented in the presence of two equivalents of n-butyllithium to generate a pentafluoroenolate. Next, treatment of the intermediate enolate with a Grignard reagent causes an addition-elimination sequence to form another enolate that is trapped with benzoyl chloride. In order to exploit the versatility of this chemistry, it is noted that the highly flammable n-butyllithium can be exchanged with (i-Pr)₂NMgCl·LiCl, a safer alternative. The intermediate magnesium pentafluoropropen-2-olate can be intercepted with a Grignard reagent and the resultant enolate can be fluorinated with an electrophilic fluorination reagent, such as NFSI. The product is a pentafluoro-gem-diol (i.e., hydrated form of the pentafluoroketone). This gem-diol is treated with the disclosed reducing conditions, described in Example 1, to make the pentafluoroisopropanols.

The production of chiral fluoroalcohols is more challenging than the non-fluorinated counterparts because the presence of fluorine confounds the determination of absolute stereochemical configuration. X-ray analysis can assist with characterization (Schemes 2A-2B). Racemic alcohols were synthesized and separated by enantiomeric resolution (i.e., hydrolysis of acetate esters).

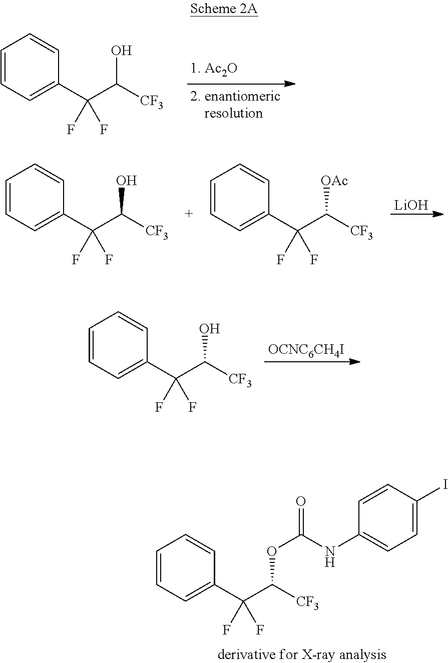

### Enantiomeric Resolution of Pentafluoroisopropanols

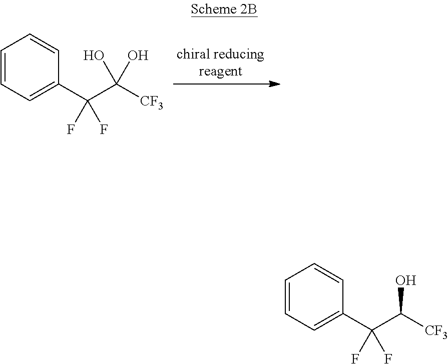 [SMILES: OC(O)(C(F)(F)F)C(F)(F)c1ccccc1 ; O[C@@H](C(F)(F)F)C(F)(F)c1ccccc1]

### Asymmetric Synthesis of Pentafluoroisopropanols

An additional synthesis route using palladium-catalyzed couplings has also been pursued. Difluoroenolates can be trapped as difluoroenoxysilanes and arylated with palladium catalyst. The enolate derived from hexafluoroisopropanol can be exploited using a similar palladium coupling (Scheme 3). Other palladium couplings designed for fluoroenolates may allow elimination of the silane, especially if the enolate can be alkylated.

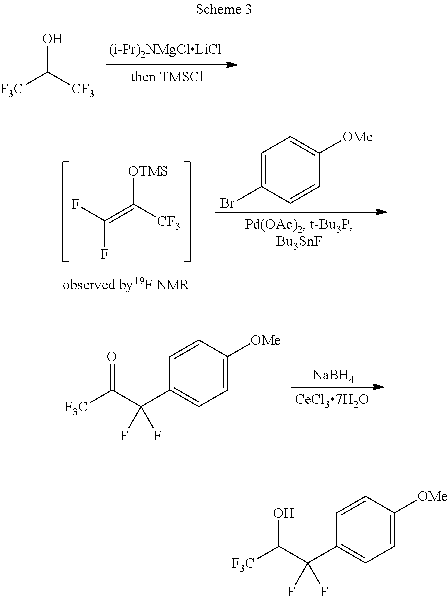

### Alternative Synthesis Strategy for Pentafluoroisopropanols

### Example 4: Production of Deuterated Solvents

Deuterated solvents are valuable as solvents for NMR spectroscopy and as tools for probing the mechanisms of reactions. The synthesis of difluorodideuteroethanols was previously reported. Pentafluoroisopropanols have been converted into fully deuterated derivatives, in an analogous fashion, using deuterated starting materials and finishing the routes described in Examples 1-3 by treatment with NaBD₄.

### Example 5: Properties of Pentafluoroisopropanols

The presence of the additional fluorines lowers boiling points compared to the non-fluorinated parent solvents. For example, the boiling point of ethanol is 78° C. and isopropanol is 83° C., whereas the boiling point to trifluoroethanol is 73° C. and hexafluoroisopropanol is 58° C. Although adding additional functional groups and molecular weight raises boiling points, the fluorines temper this effect, especially in the case of the pentafluoroisopropanols.

The solvent hybrids of pentafluoroisopropanol are especially appealing as hexafluoroisopropanol is an established co-solvent with most common organic solvents. All of the pentafluoroisopropanols were first produced as racemic mixtures; however, the isolation of enantiopure solvents was pursued and is described previously. Hexafluoroisopropanol is an established hydrogen-bond donor, which provides an ideal foundation for further work with pentafluoroisopropanols.

### Example 6: Reduction of Pentafluoro-gem-Diols to Pentafluoroisopropanols

Use of Reducing Agents: To convert a pentafluoro-gem-diol into a pentafluoroisopropanols, one starting point is to attempt reduction using reducing agents. Reducing agents such as NaBH₄ were initially employed to reduce the pentafluoro-gem-diols. NaBH₄ is commonly used with polar protic solvents, such as MeOH and EtOH. However, during this investigation, it was discovered that alcohols convert pentafluoro-gem-diols into pentafluorohemiketals. Therefore, the use of alcohol was avoided while employing reducing agents. The use of reducing agents in different solvents was also explored. Solvents such as THF with LiBH₄ or LiAlH₄ did not yield the expected transformation. Heating the pentafluoro-gem-diols at 100° C. in dioxane with LiAlH₄ returned unreacted starting materials.

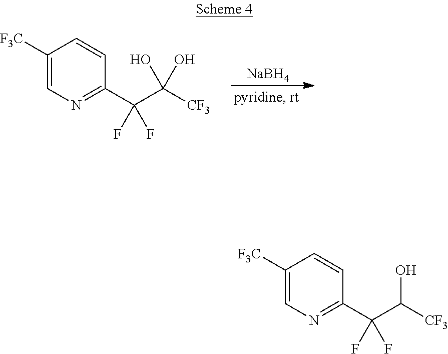 [SMILES: OC(O)(C(F)(F)F)C(F)(F)c1ccc(C(F)(F)F)cn1 ; "NaBH4" ; OC(C(F)(F)F)C(F)(F)c1ccc(C(F)(F)F)cn1]

Strategy for the reduction of a pentafluoro-gem-diol to a pentafluoroisopropanol using NaBH₄ in pyridine

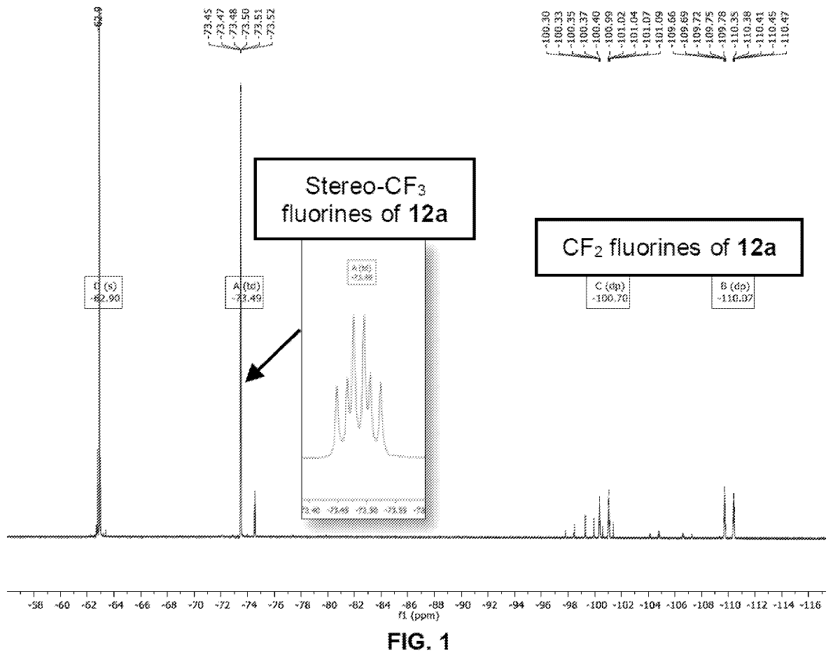

When pyridine with NaBH₄ was employed, no product formation was observed (Scheme 4). The pentafluoro-gem-diol underwent trifluoroacetate release and was consumed resulting in the formation of a difluoroketone. It is noteworthy that only pyridine did not induce trifluoroacetate release. The trifluoroacetate release occurred when a combination of pyridine and NaBH₄ was employed. However, when an additive was introduced in the reaction (Scheme 5), namely CeCk: 7H₂O, which is commonly used in Luche reduction, fluorine NMR signals were observed, corresponding to the anticipated product (FIG. 1). Subsequently, confirmation of the desired product's presence in the crude reaction was obtained through mass spectrometry.

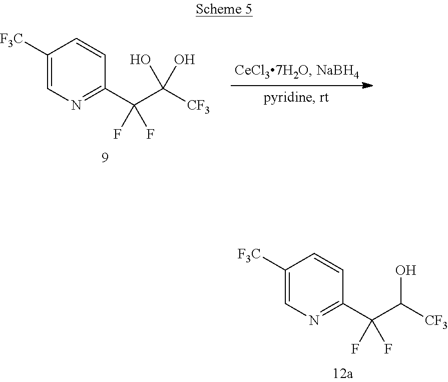 [SMILES: OC(O)(C(F)(F)F)C(F)(F)c1ccc(C(F)(F)F)cn1 ; OC(C(F)(F)F)C(F)(F)c1ccc(C(F)(F)F)cn1]

Conversion of a pentafluoro-gem-diol 9 to a pentafluoroisopropanol 12a using pyridine with CeCl₃·7H₂O and NaBH₄

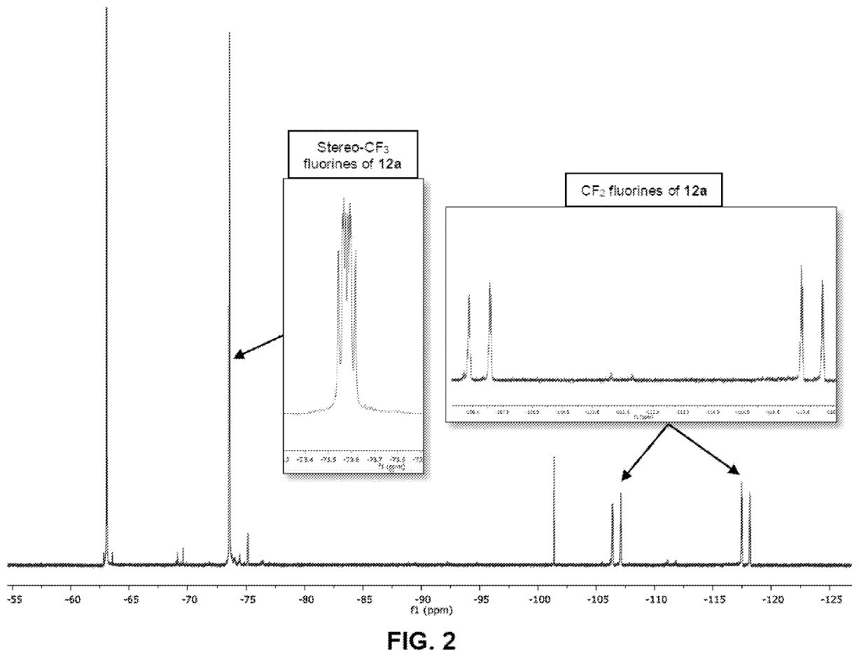

We obtained similar results with DMSO in conjunction with CeCl₃·7H₂O and NaBH₄. Other additive such as LiBr with NaBH₄ in DMSO also seemed to work. Other reducing agent such as LiAlH₄ with CeCl₃·7H₂O in DMSO did not work. The reducing agent LiBH₄ with CeCl₃·7H₂O in DMSO converted a pentafluoro-gem-diol into a pentafluoroisopropanol. Nevertheless, the challenge arose when attempting to remove pyridine or DMSO from the reaction mixture. Consequently, alternative solvents were explored in conjunction with CeCl₃·7H₂O and NaBH₄ for the reduction of pentafluoro-gem-diols. CeCl₃·7H₂O and NaBH₄ with diethyl ether or acetonitrile did not produce the expected product. In THF with CeCl₃·7H₂O and NaBH₄, a pentafluoroisopropanol was also observed, but some starting materials remained unreacted. It was discovered that a polar protic solvent such as tert-butanol did not convert gem-diols into hemiketals. Therefore, tert-butanol could serve as a viable solvent in the reaction, as it could be easily removed. The formation of a pentafluoroisopropanol was observed in tert-butanol with CeCl₃·7H₂O and NaBH₄ from a pentafluoro-gem-diol (Scheme 6). FIG. 2 shows the crude fluorine NMR of the conversion of a pentafluoro-gem-diol 9 to a pentafluoroisopropanol 12a using tert-butanol in conjunction with CeCl₃·7H₂O and NaBH₄.

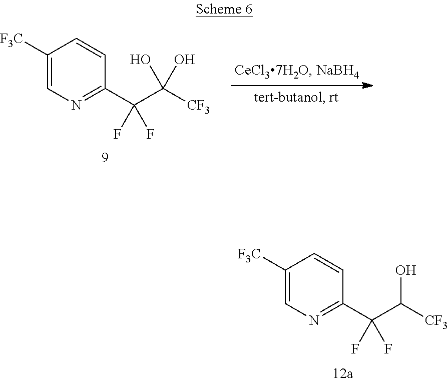 [SMILES: OC(O)(C(F)(F)F)C(F)(F)c1ccc(C(F)(F)F)cn1 ; OC(C(F)(F)F)C(F)(F)c1ccc(C(F)(F)F)cn1]

Conversion of a pentafluoro-gem-diol 9 to a pentafluoroisopropanol 12a using tert-butanol with CeCl₃·7H₂O and NaBH₄

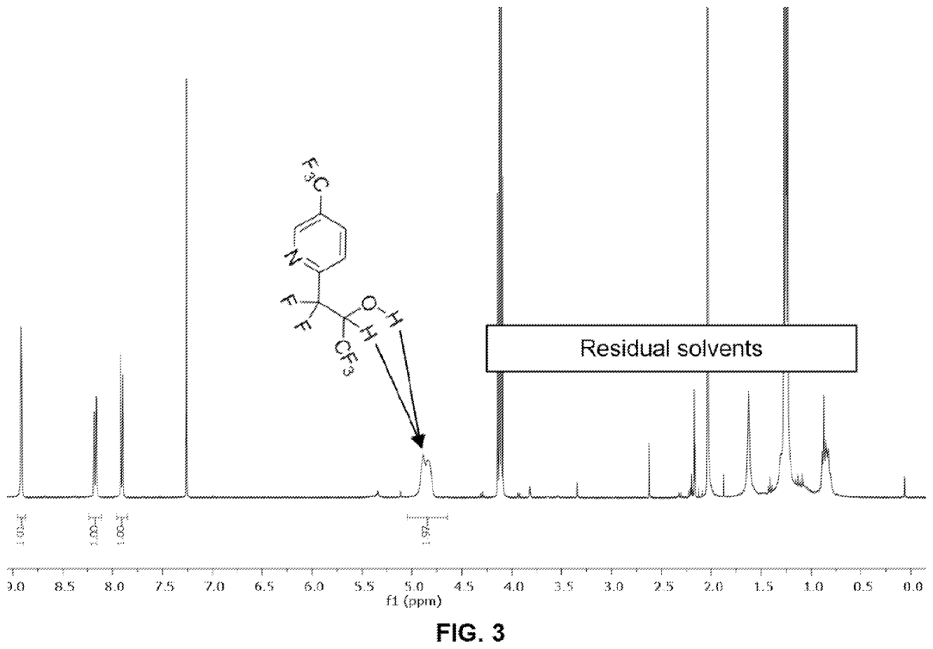

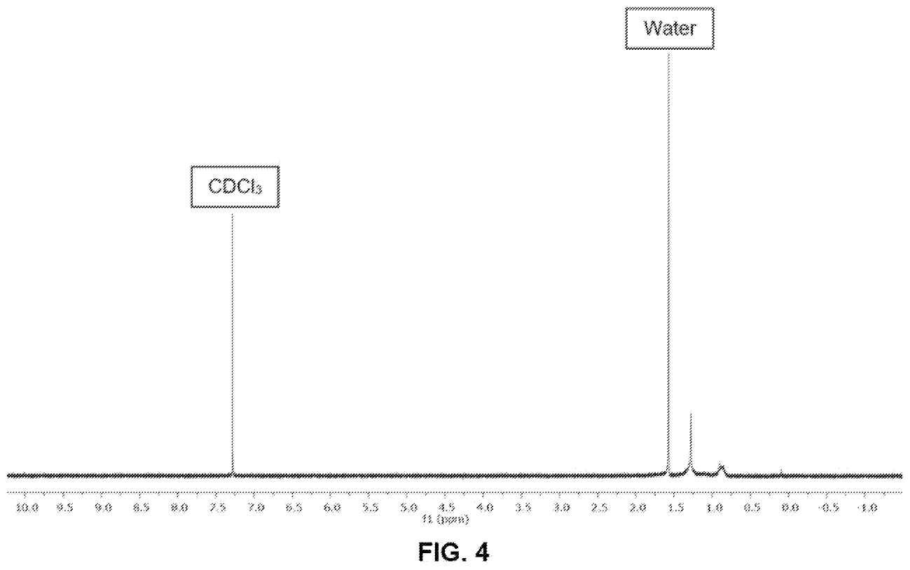

After concentrating the crude reaction mixture containing tert-butanol, flash chromatography was conducted using hexanes/ethyl acetate (8/2) as the solvent system. The eluate containing the desired compound was subjected to air drying. Proton (FIG. 3) and fluorine NMR spectra confirmed the presence of the desired compound in the air-dried sample with residual solvent peaks e.g., tert-butanol, hexanes, and ethyl acetate. However, during rotary evaporation of the air-dried sample, the compound completely evaporated, underscoring its volatile nature. FIG. 4 shows the proton NMR of the sample after rotary evaporation. This outcome is a positive and exciting development, suggesting the creation of a novel, highly volatile fluorinated alcohol, indicative of a potential new class of reagents. Now, the method only needs an efficient distillation procedure to separate the desired volatile compound from the crude mixture.

Conclusion: HFIP is a valuable solvent in organic synthesis. Derivatives of HFIP were synthesized by replacing one of the fluorines with an aryl group. The resulting pentafluoroisopropanols will represent a novel class of fluorinated alcohols. The reagents and conditions were identified as described above to synthesize pentafluoroisopropanols from pentafluoro-gem-diols. The formation of a pentafluoroisopropanol from a pentafluoro-gem-diol was achieved in tert-butanol with CeCl₃·7H₂O and NaBH₄. The resulting pentafluoroisopropanol was determined to be volatile. An efficient distillation process is required to separate pentafluoroisopropanol from the crude reaction mixture. All pentafluoro-gem-diols with an aryl group are expected to be compatible with the reaction conditions.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Adam, A. T.; et al, Synthesis of β-Fluoro-α,β-Unsaturated Amides from the Fragmentation of Morpholine 3,3,3-Trifluoropropanamide by Grignard Reagents. Org. Lett. 2020, 22, 2630-2633. PubMed PMID: 32181669; PubMed Central PMCID: PMC7465497.
2. Alexanian, E. J. et al, Org. Lett. 2019, 21, 9268-9271.
3. An, X.; et al. Fluorinated Alcohols: Magic Reaction Medium and Promoters for Organic Synthesis. Chem. Rec. 2020, 20 (2), 142-161.
4. Andersen, T. L.; et al, Direct Access to α,α-Difluoroacylated Arenes by Palladium-Catalyzed Carbonylation of (Hetero) Aryl Boronic Acid Derivatives. Angew. Chem., Int. Ed. 2016, 55, 10396-10400.
5. Atzrodt, J. et al, Angew. Chem. Intl. Ed., 2018, 57, 1758-1784.
6. Bae, J. W.; et al. Reduction of Ketones to Alcohols Using a Decaborane/Pyrrolidine/Cerium (III) Chloride System in Methanol. Tetrahedron Lett. 2001, 42 (11), 2137-2139.
7. Balnaves, A. S. et al, J. Chem. Soc., Perkins Trans. 1, 1999, 2525-2535.
8. Banphavichit, V.; et al. A Convenient Synthesis of N-Boc-Protected A-Aminonitriles from A-Amidosulfones. Synth. Commun. 2004, 34 (17), 3147-3160.
9. Barcan, G. A.; et al. Nucleophilic Aromatic Substitutions of 2-Halo-5-(Sulfamoyl) Benzoic Acids and N, O-Bis-Alkylation via Phase Transfer Catalysis: Synthesis of RoRy Inverse Agonist GSK2981278A. Org. Process Res. Dev. 2019, 23 (7), 1396-1406.
10. Baskakis, C.; et al, Synthesis of Polyfluoro Ketones for Selective Inhibition of Human Phospholipase A2 Enzymes. J. Med. Chem. 2008, 51, 8027-8037.
11. Becke, A. D. J. Chem. Phys., 2014, 140, 18A301.
12. Bellotti, P.; et al, Visible-Light Photocatalyzed peri-(3+2) Cycloadditions of Quinolines. J. Am. Chem. Soc. 2022, 144, 15662-15671.
13. Boeckman, R. K.; et al. Scalable Synthesis of (−)-Rasfonin Enabled by a Convergent Enantioselective α-Hydroxymethylation Strategy. Org. Lett. 2018, 20 (16), 5062-5065.
14. Böhm, H.-J.; et al. Fluorine in Medicinal Chemistry. ChemBioChem 2004, 5 (5), 637-643.
15. Bur, S. K. et al, Chem. Rev., 2004, 104, 2401-2432.
16. Call, A.; et al, Carboxylic Acid Directed γ-Lactonization of Unactivated Primary C—H Bonds Catalyzed by Mn Complexes: Application to Stereoselective Natural Product Diversification. J. Am. Chem. Soc. 2022, 144, 19542-19558.
17. Carreño, M. C.; et al. Oxidative De-aromatization of Para-Alkyl Phenols into Para-Peroxyquinols and Para-Quinols Mediated by Oxone as a Source of Singlet Oxygen. Angew. Chemie Int. Ed. 2006, 45 (17), 2737-2741.
18. Cerfontaine, P.; et al, Efficient Multikilogram Synthesis of a VLA-4 Antagonist via a Povarov Reaction. Org. Process Res. Dev. 2020, 24, 807-816.
19. Charaschanya, M.; et al, An Interrupted Schmidt Reaction: C—C Bond Formation Arising from Nitrilium Ion Capture. Org. Lett. 2018, 20, 6354-6358.
20. Charaschanya, M.; et al, Reagent-Controlled Regiodivergent Ring Expansions of Steroids. Nat. Commun. 2018, 9, 934.
21. Chatupheeraphat, A.; et al, Chemo- and Regioselective Magnesium-Catalyzed ortho-Alkenylation of Anilines. Org. Lett. 2019, 21, 9153-9157.
22. Chen, M.; et al, Rapid and Efficient Trifluoromethylation of Aromatic and Heteroaromatic Compounds Using Potassium Trifluoroacetate Enabled by a Flow System. Angew. Chem., Int. Ed. 2013, 52, 11628-11631.
23. Choi, K.; et al, Palladium-Catalyzed Aryldifluoromethylation of Aryl Halides with Aryldifluoromethyl Trimethylsilanes. Angew. Chem., Int. Ed. 2022, 61, e202208204.
24. Dandia, A.; et al, 2,2,2-Trifluoroethanol as Green Solvent in Organic Synthesis: A Review. Mini Rev. Org. Chem. 2014, 11, 462-476.
25. Denmark, S. E.; et al, On the Absolute Configurational Stability of Bromonium and Chloronium Ions. J. Am. Chem. Soc. 2010, 132, 1232-1233.
26. Douglas, J. J.; et al, Preparative Scale Demonstration and Mechanistic Investigation of a Visible Light-Mediated Radical Smiles Rearrangement. Org. Proc. Res. Develop. 2016, 20, 1148-1155.
27. Fini, F.; et al. An Easy Entry to Optically Active α-Amino Phosphonic Acid Derivatives Using Phase-Transfer Catalysis (PTC). Chem. Commun. 2008, No. 36, 4345.
28. Forrester, J. et al, J. Chem. Soc. Perkin Trans. 1, 1995, 18, 2289-2291.
29. Frisch, M. J. et al, Gaussian 09, Revision D.01. Gaussian, Inc., Wallingford CT, 2013.
30. Fu, W. C.; et al, Deuteriodifluoromethylation and gem-Difluoroalkenylation of Aldehydes Using CICF2H in Continuous Flow. Angew. Chem. Int. Ed. 2020, 59, 13885-13890.
31. Funabiki, K.; et al. Convenient, Functional Group-Tolerant, Transition Metal-Free Synthesis of Aryl and Heteroaryl Trifluoromethyl Ketones with the Use of Methyl Trifluoroacetate. Org. Biomol. Chem. 2018, 16 (6), 913-918.
32. Gant, T. G. J. Med. Chem., 2014, 57, 3595-3611.
33. Garcia-Losada, P.; et al, Practical Asymmetric Fluorination Approach to the Scalable Synthesis of New Fluoroaminothiazine BACE Inhibitors. Org. Process Res. Dev. 2018, 22, 650-654.
34. Ge, S.; et al, Pd-Catalyzed α-Arylation of Trimethylsilyl Enolates of α,α-Difluoroacetamides. J. Am. Chem. Soc. 2014, 136, 14401-14404.
35. Geri, J. B.; et al, Borazine-CF₃-Adducts for Rapid, Room Temperature, and Broad Scope Trifluoromethylation. Angew. Chem., Int. Ed. 2018, 57, 1381-1385.
36. Geri, J. B.; et al, The Difluoromethyl Group as a Masked Nucleophile: A Lewis Acid/Base Approach. J. Am. Chem. Soc. 2018, 140, 9404-9408.
37. Ghosh, S.; et al, HFIP-Assisted Single C—F Bond Activation of Trifluoromethyl Ketones using Visible-Light Photoredox Catalysis. Angew. Chem., Int. Ed. 2022, 61, e202115272.
38. Gillis, E. P.; et al. Applications of Fluorine in Medicinal Chemistry. J. Med. Chem. 2015, 58 (21), 8315-8359.
39. Goodman, C. G.; et al. Asymmetric Synthesis of Anti-β-Amino-α-Hydroxy Esters via Dynamic Kinetic Resolution of β-Amino-α-Keto Esters. Org. Lett. 2013, 15 (10), 2446-2449.
40. Guo, Y.; et al, Effect of Fluorine on Palladium-Catalyzed Cross-Coupling Reactions of Aryl Bromides with Trifluoromethyl Aryl Ketones via Difluoroenol Silyl or Monofluoroenol Silyl Ethers. Chem. Commun. 2007, 3583-3585.
41. Han, C. Discovery of the Trifluoroacetate Release Process and Its Strategic Application Toward the Synthesis of Biologically Active Molecules. Ph.D. Dissertation, Purdue University: West Lafayette, IN, 2013.
42. Han, C.; et al. Cleavage of Carbon-Carbon Bonds through the Mild Release of Trifluoroacetate: Generation of α,α-Difluoroenolates for Aldol Reactions. J. Am. Chem. Soc. 2011, 133 (15), 5802-5805.
43. Haranahalli, K.; et al. Recent Progress in the Strategic Incorporation of Fluorine into Medicinally Active Compounds. J. Fluor. Chem. 2019, 217, 29-40.
44. Hazlitt, R. A.; et al, Optimized Synthesis of a Pentafluoro-gem-diol and Conversion to a CF₂Br-Glucopyranose through Trifluoroacetate-Release and Halogenation. Tetrahedron Lett. 2016, 57, 1906-1908. PubMed PMID: 27182091; PubMed Central PMCID: PMC4863236.
45. Hazlitt, R. A.; et al. Generation of Magnesium Pentafluoropropen-2-Olate from Hexafluoroisopropanol and Synthesis of 2,2,4,4,4-Pentafluoro-3,3-Dihydroxyketones. J. Org. Chem. 2017, 82 (4), 2231-2236.
46. He, W.; et al. A Jak2-Selective Inhibitor Potently Reverses the Immune Suppression by Modulating the Tumor Microenvironment for Cancer Immunotherapy. Biochem. Pharmacol. 2017, 145, 132-146.
47. Hirano, T.; et al, Heterotactic Poly(N-isopropylacrylamide) Prepared via Radical Polymerization in the Presence of Fluorinated Alcohols. Polymer 2007, 48, 4921-4925.
48. Hong, D.-P.; et al, Clustering of Fluorine-Substituted Alcohols as a Factor Responsible for Their Marked Effects on Proteins and Peptides. J. Am. Chem. Soc. 1999, 121, 8427-8433.
49. Hoye, T. R.; et al, Long-Range Shielding Effects in the 1H NMR Spectra of Mosher-like Ester Derivatives. Org. Lett. 2010, 12, 1768-1771.
50. Hu, X.-S.; et al. A Highly Efficient Gold (I)-Catalyzed Mukaiyama-Mannich Reaction of α-Amino Sulfones with Fluorinated Silyl Enol Ethers To Give α-Amino α-Fluorinated Ketones. Synlett 2017, 28 (16), 2194-2198.
51. Huang, L.; et al. Catalytic Asymmetric Synthesis of Trisubstituted Aziridines. J. Am. Chem. Soc. 2011, 133 (23), 8892-8895.
52. Islam, B.; et al. Transient Formation of Hemiketals from Pentafluoro-Gem-Diols in the Presence of Alcohols. J. Fluor. Chem. 2023, 110162.
53. Ispizua-Rodriguez, X.; et al, Copper-Catalyzed Synthesis of Difluoromethyl Alkynes from Terminal and Silyl Acetylenes. J. Org. Chem. 2023, 88, 1194-1199.
54. Ji, Y.; Brueckl, T.; et al, Innate C—H Trifluoromethylation of Heterocycles. Proc. Natl. Acad. Sci. U.S.A. 2011, 108, 14411-14415.
55. Jia, H.; et al, Trifluoromethyl Thianthrenium Triflate: A Readily Available Trifluoromethylating Reagent with Formal CF3+, CF3⋅, and CF₃— Reactivity. J. Am. Chem. Soc. 2021, 143, 7623-7628.
56. Jiao, Z.; et al, Palladium-Catalyzed Enantioselective α-Arylation of α-Fluoroketones. J. Am. Chem. Soc. 2016, 138, 15980-15986.
57. Jiménez, O.; et al. A New, Mild, and Efficient Synthesis of 2,2-Difluoro-3-Hydroxyacids through a Selective Haloform Reaction. J. Org. Chem. 2005, 70 (26), 10883-10885.
58. John, J. P.; et al. Synthesis of α-Halo-α,α-Difluoromethyl Ketones by a Trifluoroacetate Release/Halogenation Protocol. J. Org. Chem. 2011, 76 (21), 9163-9168.
59. Jones-Mensah, E.; et al. Dimethyl Sulfoxide as a Synthon in Organic Chemistry. Synthesis (Stuttg). 2016, 48 (10), 1421-1436.
60. Karuturi, D.; et al. Dimroth Rearrangement to 2-N-Alkylated Amino Pyridines: An Unusual Reaction of Primary Amines with Ylidineketonitriles. Synth. Commun. 2022, 52 (3), 433-444.
61. Kashikura, W.; et al. Chiral Phosphoric Acid Catalyzed Enantioselective Synthesis of β-Amino-α,α-Difluoro Carbonyl Compounds. Org. Lett. 2011, 13 (7), 1860-1863.
62. Kawagoe, F.; et al, Recent Developments for Introducing a Hexafluoroisopropanol Unit into the Vitamin D Side Chain. J. Steroid Biochem. Mol. Biol. 2018, 177, 250-254.
63. Khaksar, S., Fluorinated Alcohols: A Magic Medium for the Synthesis of Heterocyclic Compounds. J. Fluorine Chem. 2015, 172, 51-61.
64. Khatri, H. R.; et al. Controlling the Cleavage of Carbon-Carbon Bonds To Generate α,α-Difluorobenzyl Carbanions for the Construction of Difluoromethylbenzenes. J. Org. Chem. 2019, 84 (18), 11665-11675.
65. Khatri, H. R.; et al. Generation of Formaldehyde and Formaldehyde-d 2 for Hydroxymethylations and Hydroxydeuteromethylations of Difluoroenolates and Difluorobenzyl Carbanions. Chem. Commun. 2022, 58 (36), 5490-5493.
66. King, J. F. et al, J. Org. Chem., 1998, 63, 808-811.
67. Koley, S.; et al, Cu(II)-Catalyzed Unsymmetrical Dioxidation of gem-Difluoroalkenes to Generate α,α-Difluorinated-α-phenoxyketones. J. Org. Chem. 2022, 87, 10710-10725.
68. Kutwal, M. S.; et al. Catalytic Regioselective γ-Methylenation of α,β-Unsaturated Aldehydes Using Formaldehyde via Vinylogous Aldol Condensation. Org. Lett. 2019, 21 (8), 2509-2513.
69. Kuznetsov, D. M.; et al, Acetal-induced Cationic Polymerization of Styrene and Indene in Hexafluoroisopropanol. Mendeleev Commun. 2013, 23, 274-276.
70. Lateef, H.; et al. Synthesis of Fluorinated Hemiketals from the Reaction between Alcohols and Hexafluoroacetone. J. Fluor. Chem. 2000, 104 (2), 167-171.
71. Leng, D. J.; et al, One-Pot Synthesis of Difluoromethyl Ketones by a Difluorination/Fragmentation Process. Org. Biomol. Chem. 2016, 14, 1531-1535.
72. Li, B.; et al, Transient-Ligand-Enabled ortho-Arylation of Five-Membered Heterocycles: Facile Access to Mechanochromic Materials. Angew. Chem. Int. Ed. 2018, 57, 3401-3405.
73. Li, F.; et al. Catalytic Stereoselective Cascade Reactions of Quinols with Trifluoromethyl Ketones: Direct Access to CF 3-Containing 1,3-Dioxolanes. Org. Biomol. Chem. 2016, 14 (16), 3981-3988.
74. Li, J.; et al, HFIP-Catalyzed Difluoroalkylation of Propargylic Alcohols to Access Tetrasubstituted Difluoroalkyl Allenes. Org. Lett. 2021, 23, 7264-7269.
75. Li, J.; et al, HFIP-Catalyzed Direct Dehydroxydifluoroalkylation of Benzylic and Allylic Alcohols with Difluoroenoxysilanes. ChemComm 2021, 57, 1050-1053.
76. Li, Q.; et al. New Methodology toward a, β-Unsaturated Carboxylic Acids from Saturated Acids. Org. Lett. 2014, 16 (5), 1382-1385.
77. Li, W.; et al. Visible-Light-Induced Direct C(Sp3)-H Difluromethylation of Tetrahydroisoquinolines with the in Situ Generated Difluoroenolates. Chem. Commun. 2014, 50 (56), 7521-7523.
78. Li, Y.; et al, Design, Synthesis and Evaluation of Novel 19F Magnetic Resonance Sensitive Protein Tyrosine Phosphatase Inhibitors. MedChemComm 2016, 7, 1672-1680.
79. Liu, C.; et al. Advances in Chemocatalytic Asymmetric Baeyer-Villiger Oxidations. Adv. Synth. Catal. 2020, 362 (5), 1015-1031.
80. Lu, L.; et al, HNO3/HFIP: A Nitrating System for Arenes with Direct Observation of IT-Complex Intermediates. Org. Lett. 2018, 20, 3197-3201.
81. Luan, S.; et al, Electrochemical Difluoromethylation of Electron-Rich Olefins. Org. Lett. 2023, 25, 1678-1682.
82. Maji, A.; et al, Directing Group Assisted meta-Hydroxylation by C—H Activation. Chem Sci. 2016, 7, 3147-3153.
83. Marin-Valls, R. K. et al, ACS Catal., 2019, 9, 7568-7577.
84. Masciadri, R.; et al, Regioselective Friedel-Crafts Alkylation of Anilines and Amino-Substituted Heteroarenes with Hexafluoroacetone Sesquihydrate. Eur. J. Org. Chem. 2003, 2003, 4286-4291.
85. Matsumoto, A.; et al. Organocatalytic Enantio- and Diastereoselective Construction of Syn-1,3-Diol Motifs via Dynamic Kinetic Resolution of In Situ Generated Chiral Cyanohydrins. Org. Lett. 2019, 21 (8), 2688-2692.
86. Meanwell, N. A. Fluorine and Fluorinated Motifs in the Design and Application of Bioisosteres for Drug Design. J. Med. Chem. 2018, 61 (14), 5822-5880.
87. Meng, G.; et al, β- and γ-C(sp3)-H Heteroarylation of Free Carboxylic Acids: A Modular Synthetic Platform for Diverse Quaternary Carbon Centers. Angew. Chem., Int. Ed. 2023, 62, e202214459.
88. Meng, Y. et al, J. Med. Chem., 2021, 64, 925-937.
89. Miao, W. Y. et al, J. Am. Chem. Soc., 2018, 140, 880-883.
90. Min, L. et al, J. Am. Chem. Soc., 2019, 141, 15773-15778.
91. Morstein, J.; et al, Trifluoromethylation of Arylsilanes with [(phen)CuCF3]. Angew. Chem., Int. Ed. 2016, 55, 8054-8057.
92. Motiwala, H. F.; et al, Intramolecular Friedel-Crafts Acylation Reaction Promoted by 1,1,1,3,3,3-Hexafluoro-2-propanol. Org. Lett. 2015, 17, 5484-5487.
93. Motiwala, H. F.; et al. HFIP in Organic Synthesis. Chem. Rev. 2022, 122 (15), 12544-12747.
94. Motohashi, H. et al, Org. Lett. 2018, 20, 5340-5343.
95. Mullard, A., Deuterated Drugs Draw Heavier Backing. Nat. Rev. Drug Discov. 2016, 15, 219-221.
96. Nairoukh, Z.; et al. The Formation of All-Cis-(Multi) Fluorinated Piperidines by a Dearomatization-Hydrogenation Process. Nat. Chem. 2019, 11 (3), 264-270.
97. Narita, T.; et al, Synthesis and Polymerization of Novel Fluorinated Acrylates and Methacrylates bearing Alkoxyl Groups Derived from Radical Addition Reaction of Perfluoroisopropenyl ester. J. Fluorine Chem. 2007, 128, 965-970.
98. Narita, T.-i.; et al, Unique Radical Addition Reactions onto Perfluoro-Enol Esters. Tetrahedron Lett. 1995, 36, 6091-6094.
99. Nguyen, A. L.; et al. Magnesium-Promoted Additions of Difluoroenolates to Unactivated Imines. J. Org. Chem. 2018, 83 (6), 3109-3118.
100. Ni, Y.; et al. Copper-Catalyzed Regioselective Intramolecular Electrophilic Sulfenoamination via Lewis Acid Activation of Disulfides under Aerobic Conditions. Org. Lett. 2018, 20 (14), 4350-4353.
101. Obach, R. S.; et al. Biosynthesis of Fluorinated Analogs of Drugs Using Human Cytochrome P450 Enzymes Followed by Deoxyfluorination and Quantitative Nuclear Magnetic Resonance Spectroscopy to Improve Metabolic Stability. Drug Metab. Dispos. 2016, 44 (5), 634-646.
102. Ojima, I. Exploration of Fluorine Chemistry at the Multidisciplinary Interface of Chemistry and Biology. J. Org. Chem. 2013, 78 (13), 6358-6383.
103. Onuska, N. P. R.; et al, Anti-Markovnikov Hydroazidation of Activated Olefins via Organic Photoredox Catalysis. Synlett 2020, 31, 55-59.
104. Othon, C. M.; et al, Solvation in protein (un) folding of melittin tetramer-monomer transition. Proc. Natl. Acad. Sci. U.S.A. 2009, 106, 12593-12598.
105. Pan, L.; et al, Palladium-Catalyzed Site-Selective Arylation of Aliphatic Ketones Enabled by a Transient Ligand. Chem. Commun. 2018, 54, 2759-2762.
106. Parsons, A. T.; et al, An Improved Process for the Preparation of a Covalent Kinase Inhibitor through a C—N Bond-Forming SNAr Reaction. Org. Process Res. Dev. 2018, 22, 898-902.
107. Pistritto, V. A.; et al, Mechanistic Investigations into Amination of Unactivated Arenes via Cation Radical Accelerated Nucleophilic Aromatic Substitution. J. Am. Chem. Soc. 2022, 144, 15118-15131.
108. Pistritto, V. A.; et al, Nucleophilic Aromatic Substitution of Unactivated Fluoroarenes Enabled by Organic Photoredox Catalysis. J. Am. Chem. Soc. 2020, 142, 17187-17194.
109. Prakash, G. K. S.; et al, Long-Lived Trifluoromethanide Anion: A Key Intermediate in Nucleophilic Trifluoromethylations. Angew. Chem., Int. Ed. 2014, 53, 11575-11578.
110. Prakash, G. K. S.; et al, Taming of Fluoroform: Direct Nucleophilic Trifluoromethylation of Si, B, S, and C Centers. Science 2012, 338, 1324-1327.
111. Prakash, G. S.; et al. Fluorine: The New Kingpin of Drug Discovery. Chim. Oggi-Chem. Today 2012, 30 (5), 1-7.
112. Qian, C.-P.; et al, Perfluoro-enolate Chemistry: Facile Generation and Unique Reactivities of Metal E-1-Propen-2-olates. Tetrahedron Lett. 1988, 29, 4119-4122.
113. Qu, C.-H.; et al. Microwave-Assisted Copper Catalysis of α-Difluorinated Gem-Diol toward Difluoroalkyl Radical for Hydrodifluoroalkylation of Para-Quinone Methides. J. Org. Chem. 2020, 85 (19), 12785-12796.
114. Rauch, M.; et al. Selective Conversion of Carbon Dioxide to Formaldehyde via a Bis(Silyl) Acetal: Incorporation of Isotopically Labeled C 1 Moieties Derived from Carbon Dioxide into Organic Molecules. J. Am. Chem. Soc. 2019, 141 (44), 17754-17762.
115. Reddy, M. K.; et al. Divergent Reactivity of Gem-Difluoro-Enolates toward Nitrogen Electrophiles: Unorthodox Nitroso Aldol Reaction for Rapid Synthesis of α-Ketoamides. Org. Lett. 2018, 20 (15), 4610-4613.
116. Riofski, M. V.; et al, Amidinate Salt of Hexafluoroacetone Hydrate for the Preparation of Fluorinated Compounds by the Release of Trifluoroacetate. Org. Lett. 2013, 15, 208-211. PubMed PMID: 23240844.
117. Saha, A.; et al, Photoinduced Regioselective Olefination of Arenes at Proximal and Distal Sites. J. Am. Chem. Soc. 2022, 144, 1929-1940.
118. Saidalimu, I.; et al, Highly Enantioselective Construction of 3-Hydroxy Oxindoles through a Decarboxylative Aldol Addition of Trifluoromethyl α-Fluorinated gem-Diols to N-Benzyl Isatins. Angew. Chem. Int. Ed. 2013, 52, 5566-5570.
119. Saidalimu, I.; et al, Organocatalytic Asymmetric Michael Addition/Carbon-Carbon Bond Cleavage of Trifluoromethyl α-Fluorinated gem-Diols to Nitroolefins. Adv. Synth. Catal. 2013, 355, 857-863.
120. Schlegel, M.; et al, Aliphatic C—H Functionalization Using Pyridine N-Oxides as H-Atom Abstraction Agents. ACS Catal. 2022, 12, 10499-10505.
121. Schmidt, C. Nat. Biotechnol., 2017, 35, 493-494.
122. Schubert, M.; et al, Powerful Fluoroalkoxy Molybdenum (V)Reagent for Selective Oxidative Arene Coupling Reaction. Angew. Chem., Int. Ed. 2014, 53, 2494-2497.
123. Shah, P.; et al. The Role of Fluorine in Medicinal Chemistry. J. Enzyme Inhib. Med. Chem. 2007, 22 (5), 527-540.
124. Sham, H. L.; et al. Pseudo-Symmetrical Difluoroketones. FEBS Lett. 1993, 329 (1-2), 144-146.
125. Sharif, E. U.; et al, Development of a Scalable and Practical Synthesis of AB928, a Dual A2a/A2b Receptor Antagonist. Org. Process Res. Dev. 2020, 24, 1254-1261.
126. Smith, D. T.; et al. Dearomatization Approach to 2-Trifluoromethylated Benzofuran and Dihydrobenzofuran Products. Org. Lett. 2017, 19 (13), 3508-3511.
127. Sowaileh, M. F.; et al. Agonists of the γ-Aminobutyric Acid Type B (GABAB) Receptor Derived from β-Hydroxy and β-Amino Difluoromethyl Ketones. Bioorg. Med. Chem. Lett. 2018, 28 (16), 2697-2700.
128. Sowaileh, M. F.; et al. Conversion of Methyl Ketones and Methyl Sulfones into α-Deutero-α,α-Difluoromethyl Ketones and α-Deutero-α,α-Difluoromethyl Sulfones in Three Synthetic Steps. Tetrahedron Lett. 2017, 58 (5), 396-400.
129. Sowaileh, M. F.; et al. Synthesis of Difluorinated Halohydrins by the Chemoselective Addition of Difluoroenolates to α-Haloketones. Org. Lett. 2021, 23 (13), 5098-5101.
130. Sukowski, V.; et al, S,O-Ligand Promoted meta-C—H Arylation of Anisole Derivatives via Palladium/Norbornene Catalysis. Angew. Chem., Int. Ed. 2022, 61, e202201750.
131. Takagi, Y.; et al, Determination of the Absolute Configuration of Partly Fluorinated Allylic Alcohols; the First Synthesis of Optically Pure 1,2-Difluoroallylic Alcohols. Tetrahedron Lett. 2000, 41, 7889-7892.
132. Tanaka, K.; et al, Hemilabile Benzyl Ether Enables γ-C(sp3)-H Carbonylation and Olefination of Alcohols. J. Am. Chem. Soc. 2019, 141, 15494-15497.
133. Tanemura, K. Reduction of Aldehydes and Ketones by a Stoichiometric Amount of NaBH4 Using a Small Amount of MeOH or EtOH. Results Chem. 2022, 4, 100486.
134. Targos, K.; et al, Unveiling Potent Photooxidation Behavior of Catalytic Photoreductants. J. Am. Chem. Soc. 2021, 143, 4125-4132.
135. Tarrant, P.; et al, The Reaction of Some 3- and 4-Fluorooexetanes with Acids. J. Fluorine Chem. 1988, 40, 201-215.
136. Thaisrivongs, S.; et al., Alpha.,.Alpha.-Difluoro-. Beta.-Aminodeoxystatine-Containing Renin-Inhibitory Peptides. J. Med. Chem. 1987, 30 (10), 1837-1842.
137. Tokala, R. D. et al, J. Org. Chem., 2019, 84, 5504-5513.
138. Veeraraghavan Ramachandran, P.; et al, Gem-Difluorinated Homoallyl Alcohols, β-Hydroxy Ketones, and syn- and anti-1,3-Diols via γ,γ-Difluoroallylboronates. Org. Lett. 2008, 10, 1195-1198.
139. Vekariya, R. H.; et al, Hexafluoro-2-propanol-Promoted Intermolecular Friedel-Crafts Acylation Reaction. Org. Lett. 2016, 18, 3534-3537.
140. Wang, D.; et al. Copper (I)-Catalyzed Asymmetric Pinacolboryl Addition of N-Boc-Imines Using a Chiral Sulfoxide-Phosphine Ligand. Org. Lett. 2015, 17 (10), 2420-2423.
141. Wang, S.; et al, Lewis Acid/Hexafluoroisopropanol: A Promoter System for Selective ortho-C-Alkylation of Anilines with Deactivated Styrene Derivatives and Unactivated Alkenes. ACS Catal. 2020, 10, 10794-10802.
142. Wang, W.; et al. Construction of N-Boc Monofluoromethyl Aryl Sulfones via Mannich Reaction of α-Amido Sulfones with Trifluoromethyl α-Fluorinated Arylsulfonyl Gem-Diols. J. Fluor. Chem. 2020, 235, 109537.
143. Wenzel, A. G.; et al. Asymmetric Catalytic Mannich Reactions Catalyzed by Urea Derivatives: Enantioselective Synthesis of β-Aryl-β-Amino Acids. J. Am. Chem. Soc. 2002, 124 (44), 12964-12965.
144. Wever, W. J.; et al, Visible Light Mediated Activation and O-Glycosylation of Thioglycosides. Org. Lett. 2013, 15, 30-33.
145. Wu, X.; et al. K2S208-Activated Friedel-Crafts Type Alkylation of Indoles with α-Amido Sulfones. Synthesis (Stuttg). 2017, 49 (22), 5017-5024.
146. Xiao, Q.; et al. 3-Difluoroalkyl Quaternary Oxindoles Inhibit Macrophage Pyroptosis by Blocking Inflammasome Recruitment of Caspase-1. ACS Med. Chem. Lett. 2020, 11 (7), 1392-1401.
147. Xie, C.; et al, Assembly of Fluorinated Quaternary Stereogenic Centers through Catalytic Enantioselective Detrifluoroacetylative Aldol Reactions. Angew. Chem. Int. Ed. 2015, 54, 6019-6023.
148. Xie, C.; et al, Asymmetric Synthesis of Quaternary α-Fluoro-β-keto-amines via Detrifluoroacetylative Mannich Reactions. Chem. Commun. 2015, 51, 9149-9152.
149. Xie, C.; et al, Generalized Access to Fluorinated β-Keto amino Compounds through Asymmetric Additions of α,α-Difluoroenolates to CF3-Sulfinylimine. Org. Biomol. Chem. 2014, 12, 7836-7843.
150. Xie, C.; et al, Operationally Convenient Method for Preparation of Sulfonamides Containing α,α-Difluoro-β-amino Carbonyl Moiety. Tetrahedron Lett. 2014, 55, 5908-5910.
151. Xie, C.; et al. Synthesis of α,α-Difluoro-β-Amino Carbonyl-Containing Sulfonamides and Related Compounds. J. Fluor. Chem. 2015, 172, 13-21.
152. Yang, J.; et al. Synthesis of 2,2-Difluoro-3-Hydroxy-1,4-Diketones via an HFIP-Catalyzed Mukaiyama Aldol Reaction of Glyoxal Monohydrates with Difluoroenoxysilanes. J. Org. Chem. 2022, 87 (2), 1144-1153.
153. Yang, K.; et al, Ligand-Promoted Palladium-Catalyzed β-Methylene C—H Arylation of Primary Aldehydes. Chem. Sci. 2022, 13, 5938-5943.
154. Yang, Y.; et al, Synthesis and Properties of Partially Fluorinated Amorphous Ring Containing Polymers: Poly[bis(2,2-difluorovinyl) formal], Poly[bis(2,2-difluorovinyl)difluoroformal], and Poly[bis(1-deuterio-2,2-difluorovinyl)difluoroformal]. Macromolecules 2004, 37, 7918-7923.
155. Yerien, D. E.; et al. Fluorination Methods in Drug Discovery. Org. Biomol. Chem. 2016, 14 (36), 8398-8427.
156. You, L.; et al. Secondary Alcohol Hemiacetal Formation: An in Situ Carbonyl Activation Strategy. Org. Lett. 2009, 11 (22), 5126-5129.
157. Zafrani, Y.; et al. Modulation of the H-Bond Basicity of Functional Groups by α-Fluorine-Containing Functions and Its Implications for Lipophilicity and Bioisosterism. J. Med. Chem. 2021, 64 (8), 4516-4531.
158. Zhang, P.; et al, Synthesis of Pentafluorinated β-Hydroxy Ketones. J. Org. Chem. 2012, 77, 8840-8844.
159. Zhang, P.; et al. Catalytic Enantioselective Difluoroalkylation of Aldehydes. Angew. Chemie Int. Ed. 2013, 52 (30), 7869-7873.
160. Zhang, Y.; et al. Fluorinated-Antioxidant Derivatives with Improved Pharmacological Activity: A Theoretical Study. J. Biomol. Struct. Dyn. 2020, 1-8.
161. Zhao, W.; et al, Fast and Selective Organocatalytic Ring-Opening Polymerization by Fluorinated Alcohol without a Cocatalyst. Nature Commun. 2019, 10, 3590.
162. Zhao, Y. et al, Theor. Chem. Acc., 2008, 120, 215-241.

1. A method for making a substituted pentafluoroisopropanol, the method comprising contacting a pentafluoro-gem-diol of Formula I with a reducing agent: 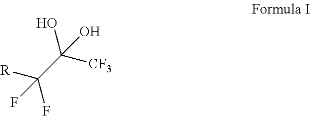
wherein R comprises a substituted or unsubstituted aryl or heteroaryl group having Formula IIa or IIb, a ketone having Formula III, a C₃-C₆ substituted or unsubstituted cycloalkyl or heterocycloalkyl group, a C₁-C₅ linear or branched alkane, a substituted or unsubstituted ether, a substituted or unsubstituted amide, a substituted or unsubstituted ester, an acetyl group, or a substituted or unsubstituted sulfoxide; 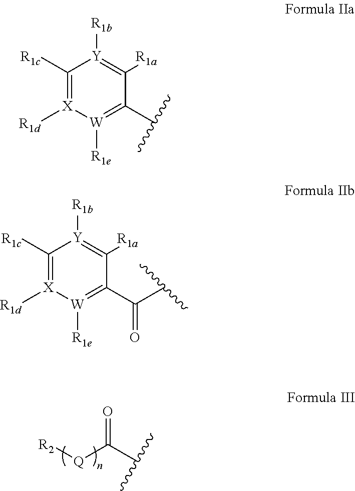
wherein, in Formula IIa or IIb, W, X, and Y are independently C or N;
wherein R₁ₐ and R_{1c} are independently selected from hydrogen, nitro, halogen, cyano, trifluoromethyl, hydroxyl, linear or branched C₁-C₁₀ alkyl, or any combination thereof;
wherein, when Y is C, R_{1b} is selected from hydrogen, nitro, halogen, cyano, trifluoromethyl, hydroxyl, linear or branched C₁-C₁₀ alkyl, or any combination thereof and when Y is N, R_{1b} is absent;
wherein, when X is C, R_{1d} is selected from hydrogen, nitro, halogen, cyano, trifluoromethyl, hydroxyl, linear or branched C₁-C₁₀ alkyl, or any combination thereof and when X is N, R_{1d} is absent; and
wherein, when W is C, R₁ₑ is selected from hydrogen, nitro, halogen, cyano, trifluoromethyl, hydroxyl, linear or branched C₁-C₁₀ alkyl, or any combination thereof and when W is N, R₁ₑ is absent;
wherein, in Formula III, n is from 0 to 10;
wherein when n is 1, Q is —CH₂—, and when n is from 2 to 10, Q is —CH₂— or —CH—; and
wherein R₂ is selected from a substituted or unsubstituted C₆-C₁₀ aryl or heteroaryl group, an adamantyl group, or any combination thereof.

2. The method of claim 1, wherein in Formula IIa or IIb, X and Y are C and W is N or C.

3. The method of claim 2, wherein W is C, wherein R₁ₐ, R_{1b}, and R₁ₐ are H, and wherein R_{1c} and R₁ₑ are independently selected from CF₃, NO₂, H, and methyl.

4. The method of claim 2, wherein W is N, wherein R₁ₐ and R_{1b} are H, and wherein R_{1c} and R_{1d} are independently selected from CF₃, NO₂, H, and methyl.

5.-6. (canceled)

7. The method of claim 1 wherein the pentafluoro-gem-diol of Formula I is selected from: 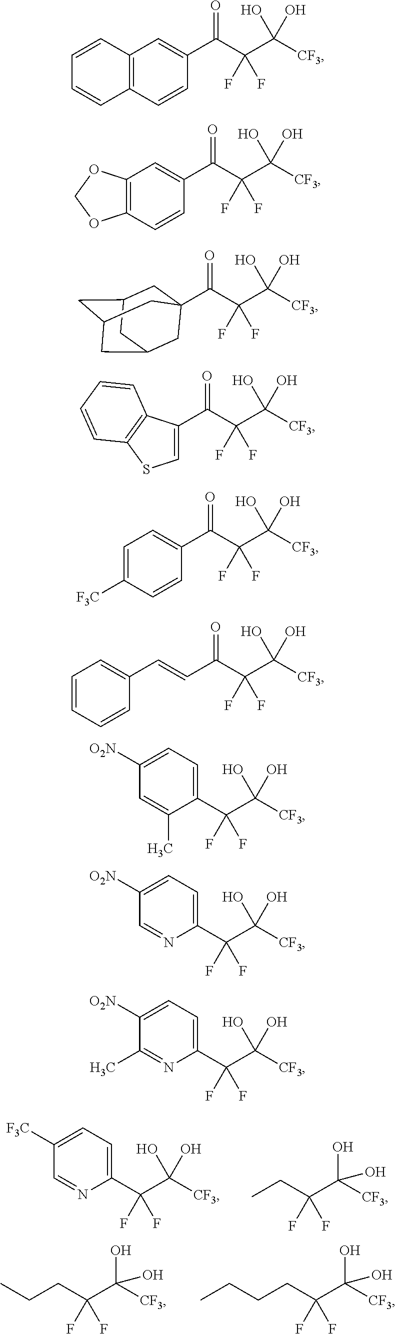 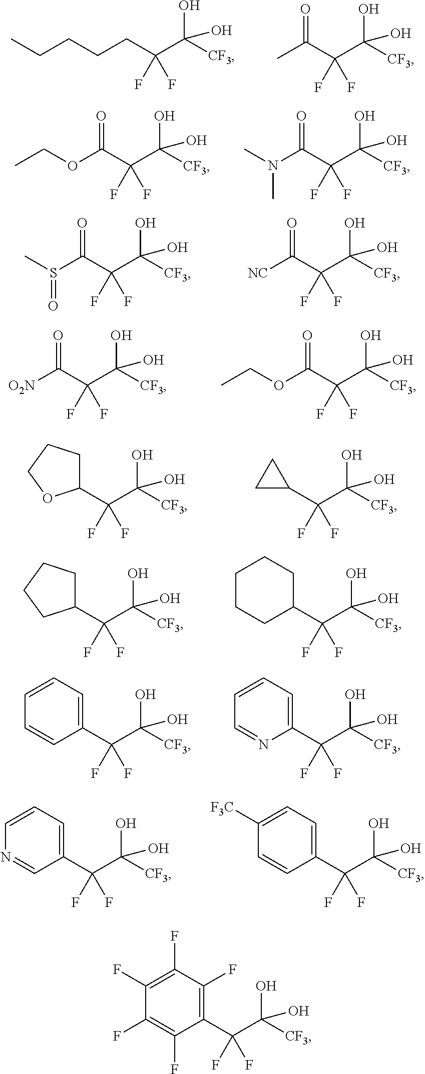
or any combination thereof.

8. The method of claim 1, wherein the reducing agent comprises NaBH₄, LiBH₄, LiAlH₄, diisobutylaluminum hydride (DIBALH), or any combination thereof.

9. The method of claim 1, further comprising contacting the pentafluoro-gem-diol of Formula I with CsCl₃ or a hydrate thereof.

10. The method of claim 1, further comprising contacting the pentafluoro-gem-diol of Formula I with a basic additive comprising triethylamine (Et₃N), 1,8-diazabicyclo[5.4.0]undec-7-ene (DBU), 1,5-diazabicyclo[4.3.0]non-5-ene (DBN), collidine, lutidine, N,N-diisopropylethylamine (iPr₂NEt), or any combination thereof.

11.-13. (canceled)

14. The method of claim 1, wherein the substituted pentafluoroisopropanol has Formula IV: 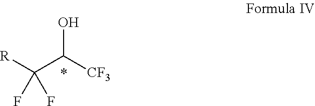
wherein the substituted pentafluoroisopropanol has R stereochemistry, S stereochemistry, or a combination of R and S stereochemistry at a carbon atom indicated by *;
wherein R comprises a substituted or unsubstituted aryl or heteroaryl group having Formula IIa or Formula IIb or a ketone having Formula III, a C₃-C₆ substituted or unsubstituted cycloalkyl or heterocycloalkyl group, a C₁-C₅ linear or branched alkane, a substituted or unsubstituted ether, a substituted or unsubstituted amide, a substituted or unsubstituted ester, an acetyl group, or a substituted or unsubstituted sulfoxide: 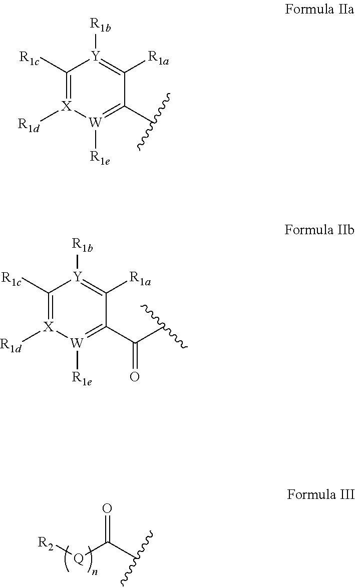
wherein, in Formula IIa and Formula IIb, W, X, and Y are independently C or N;
wherein R₁ₐ and R_{1c} are independently selected from hydrogen, nitro, halogen, cyano, trifluoromethyl, hydroxyl, linear or branched C₁-C₁₀ alkyl, or any combination thereof;
wherein, when Y is C, R_{1b} is selected from hydrogen, nitro, halogen, cyano, trifluoromethyl, hydroxyl, linear or branched C₁-C₁₀ alkyl, or any combination thereof and when Y is N, R_{1b} is absent;
wherein, when X is C, R_{1d} is selected from hydrogen, nitro, halogen, cyano, trifluoromethyl, hydroxyl, linear or branched C₁-C₁₀ alkyl, or any combination thereof and when X is N, R_{1d} is absent; and
wherein, when W is C, R₁ₑ is selected from hydrogen, nitro, halogen, cyano, trifluoromethyl, hydroxyl, linear or branched C₁-C₁₀ alkyl, or any combination thereof and when W is N, R₁ₑ is absent;
wherein, in Formula III, n is from 0 to 10;
wherein when n is 1, Q is —CH₂—, and when n is from 2 to 10, Q is —CH₂— or —CH—; and
wherein R₂ is selected from a substituted or unsubstituted C₆-C₁₀ aryl or heteroaryl group, an adamantyl group, or any combination thereof.

15. (canceled)

16. A substituted pentafluoroisopropanol having Formula IV: 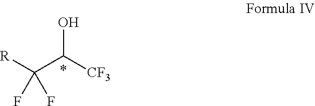
wherein the substituted pentafluoroisopropanol has R stereochemistry, S stereochemistry, or a combination of R and S stereochemistry at a carbon atom indicated by *;
wherein R comprises a substituted or unsubstituted aryl or heteroaryl group having Formula IIa or Formula IIb or a ketone having Formula III, a C₃-C₆ substituted or unsubstituted cycloalkyl or heterocycloalkyl group, a C₁-C₅ linear or branched alkane, a substituted or unsubstituted ether, a substituted or unsubstituted amide, a substituted or unsubstituted ester, an acetyl group, or a substituted or unsubstituted sulfoxide; 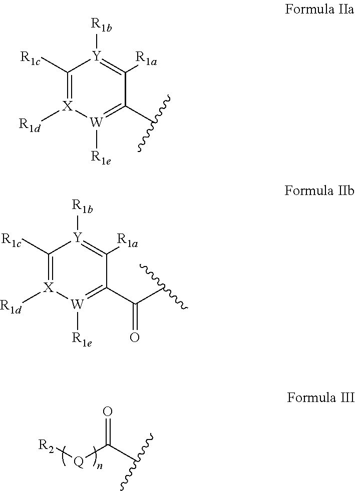
wherein, in Formula IIa and Formula IIb, W, X, and Y are independently C or N;
wherein R₁ₐ and R_{1c} are independently selected from hydrogen, nitro, halogen, cyano, trifluoromethyl, hydroxyl, linear or branched C₁-C₁₀ alkyl, or any combination thereof;
wherein, when Y is C, R_{1b} is selected from hydrogen, nitro, halogen, cyano, trifluoromethyl, hydroxyl, linear or branched C₁-C₁₀ alkyl, or any combination thereof and when Y is N, R_{1b} is absent;
wherein, when X is C, R_{1d} is selected from hydrogen, nitro, halogen, cyano, trifluoromethyl, hydroxyl, linear or branched C₁-C₁₀ alkyl, or any combination thereof and when X is N, R_{1d} is absent; and
wherein, when W is C, R₁ₑ is selected from hydrogen, nitro, halogen, cyano, trifluoromethyl, hydroxyl, linear or branched C₁-C₁₀ alkyl, or any combination thereof and when W is N, R₁ₑ is absent;
wherein, in Formula III, n is from 0 to 10;
wherein when n is 1, Q is —CH₂—, and when n is from 2 to 10, Q is —CH₂— or —CH—; and
wherein R₂ is selected from a substituted or unsubstituted C₆-C₁₀ aryl or heteroaryl group, an adamantyl group, or any combination thereof.

17. The substituted pentafluoroisopropanol of claim 16, wherein in Formula IIa and Formula IIb, X and Y are C and W is N or C.

18. The substituted pentafluoroisopropanol of claim 17, wherein W is C, wherein R₁ₐ, R_{1b}, and R_{1d} are H, and wherein R_{1c} and R₁ₑ are independently selected from CF₃, NO₂, H, and methyl.

19. The substituted pentafluoroisopropanol of claim 17, wherein W is N, wherein R₁ₐ and R_{1b} are H, and wherein R_{1c} and R_{1d} are independently selected from CF₃, NO₂, H, and methyl.

20. The substituted pentafluoroisopropanol of claim 16, wherein R is a C₁ to C₅ linear alkyl.

21. The substituted pentafluoroisopropanol of claim 16, wherein n is from 0 to 2.

22. The substituted pentafluoroisopropanol of claim 16, having the formula: 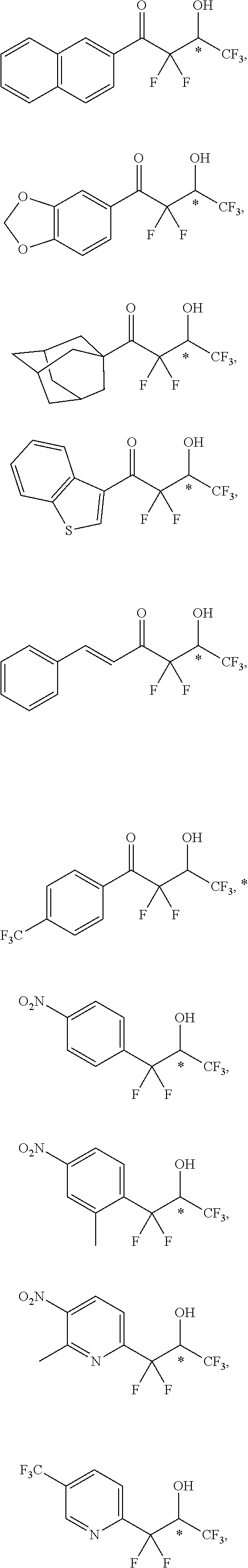 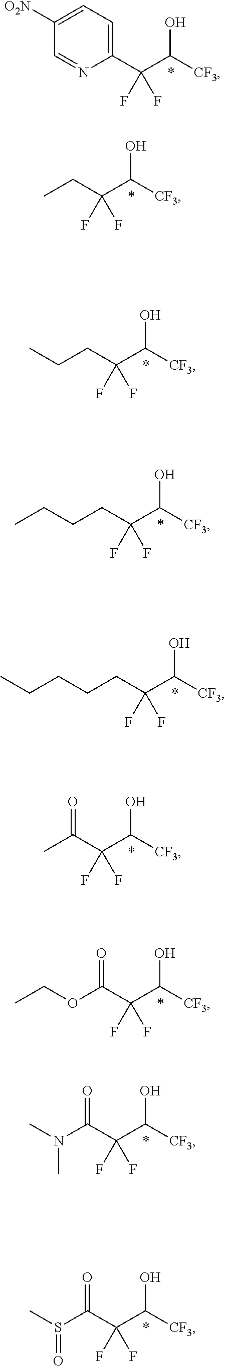 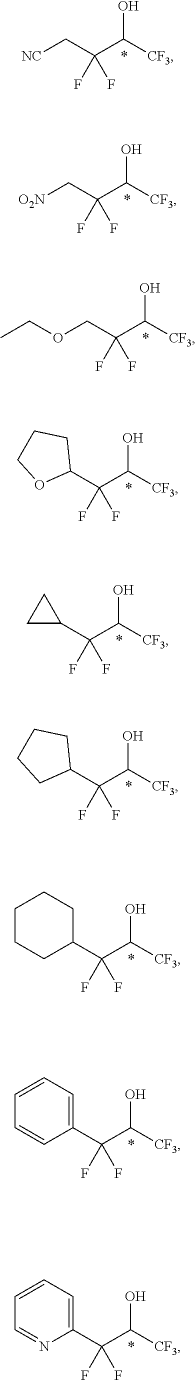 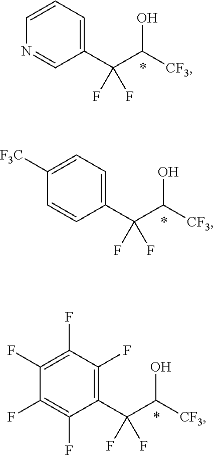 [SMILES: OC(C(F)(F)F)C(F)(F)c1cccnc1 ; OC(C(F)(F)F)C(F)(F)c1ccc(C(F)(F)F)cc1 ; OC(C(F)(F)F)C(F)(F)c1c(F)c(F)c(F)c(F)c1F]
or any combination thereof.

23. The substituted pentafluoroisopropanol of claim 16, wherein the substituted pentafluoroisopropanol comprises at least one deuterium atom in place of at least one hydrogen atom.

24. (canceled)

25. The substituted pentafluoroisopropanol of claim 16, wherein the substituted pentafluoroisopropanol has an enantiomeric excess of from about 5% to about 95% of an R enantiomer at the carbon atom indicated by *.

26. The substituted pentafluoroisopropanol of claim 16, wherein the substituted pentafluoroisopropanol has an enantiomeric excess of from about 5% to about 95% of an S enantiomer at the carbon atom indicated by *.

27. The substituted pentafluoroisopropanol of claim 16, wherein the substituted pentafluoroisopropanol is present as a racemic mixture.

28.-31. (canceled)
